(19) United States
(12) Patent Application Publication
(10) Pub. No.: US 2025/0107850 A1
(43) Pub. Date: Apr. 3, 2025

(54) Instrument Accuracy Enhancement System and Method

(71) Applicant: Rick Schue, Irwindale, CA (US)

(72) Inventor: Rick Schue, Irwindale, CA (US)

(21) Appl. No.: 18/903,844

(22) Filed: Oct. 1, 2024

Related U.S. Application Data
(60) Provisional application No. 63/541,897, filed on Oct. 1, 2023.

(51) Int. Cl. A61B 34/10; A61B 90/00

(52) U.S. Cl. CPC A61B 34/10; A61B 90/06; A61B 90/36; A61B 2090/067; A61B 2090/376

(57) ABSTRACT

An accuracy enhancement system to increase the accuracy of an alignment feedback system that measures the tilt of a surgical instrument is provided. The accuracy enhancement system also correlates tilt information measured by the surgical instrument with imaging information taken by a clinical imaging system.

### CROSS-REFERENCE TO RELATED APPLICATION

This application claims the benefit of U.S. Provisional Application No. 63/541,897, filed Oct. 1, 2023, the contents of which are incorporated herein by reference, as if fully set forth herein.

### FIELD OF THE INVENTION

This invention relates to accuracy enhancement of electrical instruments, including a system and method for improving the accuracy of electronic handheld surgical instruments.

### BACKGROUND OF THE INVENTION

Many spinal stabilization surgeries are performed every year. During the procedures, stabilizing structures, such as spinal rods and/or plates, are secured to a patient's spine by pedicle screws. The pedicle screws are implanted into the pedicles of the patient's vertebrae.

The implantation of the pedicle screws may involve first forming a pilot hole through the pedicle and into the vertebral body of the pertinent vertebrae of the patient's spine. The pedicle screw may then be implanted into the pilot hole. In order to properly place the pedicle screw and avoid damaging the patient's spinal column, the position, angular orientation, and trajectory of the pilot hole should be precisely executed.

Handheld surgical tools used to perform the above procedures, such as probes, may include an angular displacement sensor that provides real time measurements of the tool's orientation during formation of the pilot hole. The surgeon may use this information to tilt or orient the tool at the proper sagittal and axial angles, such that the pilot hole is properly oriented.

Imaging instrumentation (such as fluoroscopic imaging systems) also may be used to provide images of the patient's spine to assist in the formation of pilot holes at the proper orientation and the subsequent implantation of pedicle screws.

However, such sensors configured with the surgical tool may take angular measurements with respect to gravity while the imaging instrumentation may take images with respect to the patient's anteroposterior and/or craniocaudal axis. Because of this, the data provided by the sensors may not match the data provided by the imaging system thereby causing ambiguity regarding the accuracy of the measurements.

Accordingly, there is a need for a system and method that correlates data received from surgical tools equipped with sensors and data received from traditional clinical imaging systems. There also is a need for a system and method that corrects for the measurement deficiencies of such sensors.

### SUMMARY OF THE INVENTION

According to an aspect of the current invention, one or more embodiments are described below for a system and method for using a handheld surgical instrument comprising a measurement sensor assembly that is attached to the handheld instrument, that includes an accelerometer referenced to an X-axis, Y-axis and Z-axis, and which measures displacements of the accelerometer and handheld instrument along the X-axis, Y-axis and Z-axis in relation to gravity, and a controller or processor that receives the information regarding the gravitational field measurements in the three axes and process it to provide angular orientation information regarding the sagittal and axial displacement of the handheld surgical instrument; the method comprising: aligning the Y-axis of the measurement sensor assembly with the patient's anteroposterior axis, aligning the X-axis of the measurement sensor assembly with the patient's craniocaudal axis, tilting the handheld surgical instrument to a first axial angle preferably without rotating the measurement sensor assembly about its Y-axis, or preferably with limited amount of rotation, tilting the handheld surgical instrument to a first sagittal angle preferably without rotating the measurement sensor assembly about its Y-axis, or preferably with limited amount of rotation, using the measurement sensor assembly to measure a first X displacement value, a first Y displacement value and a first Z displacement value, calculating a first axial angle value using only the first Z displacement value and gravity, and calculating a first sagittal angle value using the first X displacement value and the first Y displacement value.

In another embodiment, the system and method further comprise using an imaging system to measure a second sagittal angle with respect to the patient's anteroposterior axis, foreshortening the first sagittal angle value by the first axial angle value to determine a foreshortened sagittal angle value, and correlating the second sagittal angle with the foreshortened sagittal angle to determine if the second sagittal angle substantially matches the foreshortened sagittal angle.

In another embodiment, the system and method further comprise, in response to a determination that the second sagittal angle substantially matches the foreshortened sagittal angle, enabling further use of the measurement sensor assembly via an application; and in response to a determination that the second sagittal angle does not substantially match the foreshortened sagittal angle, disabling further use of the measurement sensor assembly via the application.

In another embodiment, the imaging system includes a fluoroscope system, and the system may correlate angular orientation information provided by the alignment feedback system with angular orientation information provided by the fluoroscope.

In another embodiment, the method further comprises aligning the anteroposterior axis of the patient with gravity.

In another embodiment, the accelerometer includes a tri-axial accelerometer.

According to another aspect, one or more embodiments are provided below for a system and method comprising providing a sensor adapted to take angular displacement measurements relative to a three-dimensional X, Y and Z coordinate system, aligning a Y-axis of the sensor with gravity, aligning an X-axis of the sensor with a craniocaudal axis of a patient, tilting the sensor to a first sagittal angle and to a first axial angle preferably without rotating the sensor about the Y-axis, or preferably with limited rotation, using the sensor to measure a first sagittal angle and a first axial angle of the patient, each with respect to gravity, using an imaging system to measure a second sagittal angle of the patient with respect to an anteroposterior axis of the patient, foreshortening the first sagittal angle by the first axial angle to determine a foreshortened sagittal angle, and correlating the second sagittal angle with the foreshortened sagittal angle to determine if the second sagittal angle substantially matches the foreshortened sagittal angle.

In another embodiment, the system and method further comprise, in response to a determination that the second sagittal angle substantially matches the foreshortened sagittal angle, enabling further use of the sensor via an application; and in response to a determination that the second sagittal angle does not substantially match the foreshortened sagittal angle, disabling further use of the sensor via the application.

In another aspect of the current invention, a method of calibrating a measurement sensor assembly prior to its use in a surgical procedure is described.

In another aspect of the current invention, an apparatus for calibrating a measurement sensor assembly is described.

In another aspect of the invention, the system and method may include software and/or controls that limits the life angular orientation measurement system. For example, the measurement sensor assembly may be programmed so that the system will not operate if a new battery is installed.

The present invention is specified in the claims as well as in the below description.

### BRIEF DESCRIPTION OF THE DRAWINGS

Various objects, features and attendant advantages of the present invention will become fully appreciated when considered in conjunction with the accompanying drawings, in which like reference characters designate the same or similar elements throughout the several views, and wherein:

FIG. 1 shows aspects of an accuracy enhancement system according to exemplary embodiments hereof;

FIG. 2 shows aspects of an active instrument according to exemplary embodiments hereof;

FIG. 3 shows coordinate systems used by an accuracy enhancement system according to exemplary embodiments hereof;

FIGS. 3A-3B show a tilting of a measurement sensor with respect to a patient's spine according to exemplary embodiments hereof;

FIGS. 4A-4B and 5A-5B show a tilting of a measurement sensor with respect to a patient's spine according to exemplary embodiments hereof;

FIG. 6 shows a workflow of an accuracy enhancement system according to exemplary embodiments hereof;

FIG. 7 shows a calibration fixture according to exemplary embodiments hereof;

FIG. 8 shows orientations of a measurement sensor according to exemplary embodiments hereof;

FIG. 9 shows a response of a measurement sensor according to exemplary embodiments hereof;

FIG. 10 shows a response of a measurement sensor according to exemplary embodiments hereof;

FIG. 11 shows a first angle foreshortened by a second angle shows a response of a measurement sensor according to exemplary embodiments hereof; and

FIG. 12 shows an active instrument with a guide member.

FIG. 13 is a firmware architecture design chart.

FIG. 14 shows firmware code setup and loop routines.

FIG. 15 is an App architecture design chart.

FIG. 16 is a composite angle schematic.

FIG. 17 is a composite angle error table.

FIG. 18 is a chart showing angular composite error.

FIG. 19 shows fluoroscopic angle capability results.

FIG. 20 shows a relationship between the angular orientation system and operating table.

FIG. 21 shows positioning of the system.

FIG. 22 shows positioning of the system.

FIG. 23 shows positioning of the system.

FIG. 24 shows positioning of the system.

FIG. 25 shows positioning of the system.

FIG. 26 shows a MEMS accelerometer.

FIG. 27 shows an accelerometer block diagram.

FIG. 28 shows an integrated microprocessor.

FIG. 29 shows accelerometer coordinates.

FIG. 30 shows coordinates on an instrument.

FIG. 31 shows angle normalization.

### DETAILED DESCRIPTION OF THE PREFERRED EMBODIMENTS

For purposes of this specification, the term “active instrument” generally means an instrument, e.g., a handheld instrument, that may take measurement readings during use via embedded electronics, and that may provide the readings to the user (preferably in real time). An active instrument may include one or more electronic sensors that sense(s) physical inputs and that convert(s) the physical inputs into electrical outputs. For example, an active instrument designed to sense the spatial positioning of the instrument during use may include one or more embedded accelerometers. The accelerometers may take angular displacement readings and communicate them to a controller where they may be plotted, overlaid with other data, listed, or otherwise processed for display to the user. Other types of sensors also may be used (e.g., gyroscopes), and other types of feedback also may be provided, such as, without limitation, visual feedback (e.g., blinking LEDs), audio feedback (e.g., beeps), haptic feedback (e.g., vibrations), other types of feedback, and any combinations thereof.

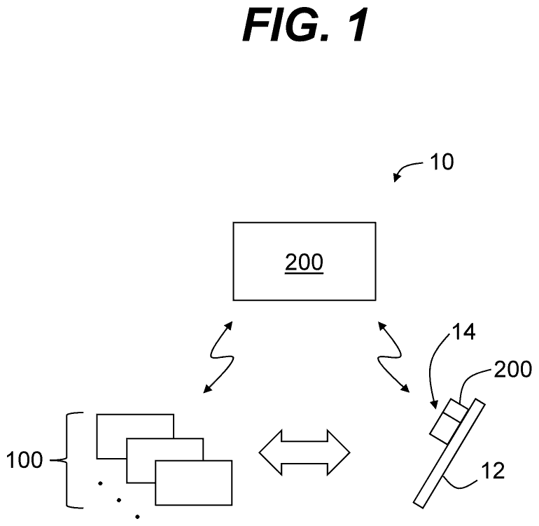

In general, as shown in FIG. 1, the system and method according to exemplary embodiments hereof provides an accuracy enhancement system 10 and method (also referred to herein as simply the system 10) for active instruments 12. In some embodiments, the system 10 includes particular instrumentation 100 and methodologies designed to enhance the accuracy of specific types of active instruments 12. The system 10 also may include a controller 200 integrated with the active instrument 12 (e.g., a microprocessor) and/or any other type(s) of controller(s) 200 (e.g., separate controllers in communication with the active instrument 12). The controller(s) 200 also may communicate with the instrumentation 100 and/or with one another (preferably wirelessly). Any of the calculations and/or data processing described herein may be performed by any of the controllers 200.

In some embodiments, the system 10 may be designed to enhance the accuracy of active instruments 12 that include one or more accelerometers implemented to measure the real time three-dimensional position, orientation, and trajectory of the active instrument 12. The accelerometer may measure the gravitational field in three axes, i.e., X-axis, Y-axis and Z-axis. The three gravitational measurements may be converted into angles by basic trigonometry by an integrated microprocessor and thus compute angles of displacement of system 14 and active instrument 12. These displacement angles in two planes may be communicated to a display device for display to a surgeon.

As such, the system 10 may serve to measure the angle of surgical instruments in two planes relative to a vertical plumb line in line with gravity. It may be used during lumbosacral pedicle screw implantation in conjunction with applicable spinal instrumentation and as an adjunct to fluoroscopy or intraoperative x-ray.

For example, the alignment feedback system 14 may be attached to a surgical instrument or tool 16, to measure the axial and sagittal angles thereof, relative to gravity. The alignment feedback system 14 may be connected to a tablet or other display device, preferably wirelessly. The axial and sagittal angular measurements may be displayed on the tablet or other display device via an Application (App) installed thereon. The App may serve as the user interface of system 10. The sensor or alignment feedback system 14 may be paired with the display device upon power-up. The sensor or system 14 may then be attached to a tool 16, e.g., inserted into a slot on tool 16, so as to create active instrument 12.

The system 10 may include programmable software in two forms: (1) programmed firmware loaded onto an integrated chip that forms system 14 and (2) software of the App that may run on a tablet or other display device. Both types of software may be summarized as follows.

The programmed firmware may be responsible for data collection, manipulation, formatting and output via Bluetooth to the App for display to the user (surgeon). The programmed firmware converts the accelerometer's measurements of the gravitational field in the X-axis, Y-axis, and Z-axis, into displacement angles, i.e., the sagittal and axial angular orientations of the sensor 14 and active instrument 12. The displacement angles are then communicated to the display device via the App run on the display device.

In some embodiments, the App need not perform any data manipulation or calculation functions once the data stream is received from the chip or alignment feedback system 14. Instead, it may function so as to display angular orientation measurements, i.e., the sagittal and axial angular orientations of the sensor 14 and active instrument 12, on the tablet or other display device.

The system 10 is designed to enhance the accuracy of this spatial information. In some embodiments, the system 10 also may correlate data taken from the active instrument 12 with data taken from other devices (e.g., fluoroscope systems) to validate the data.

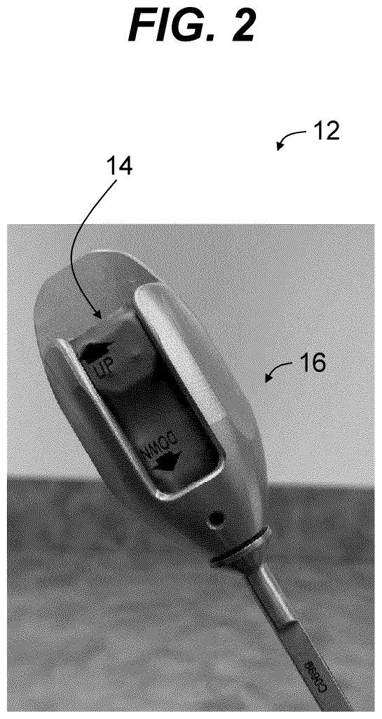

For the purposes of this specification, active instruments 12 will be described primarily as handheld surgical instruments. For example, as shown in FIG. 2, an active instrument 12 may include an orthopedic surgical tool 16, such as an awl or probe, equipped with an alignment feedback system 14. An example of the alignment feedback system 14 is described in U.S. Pat. No. 11,484,381, entitled Instrument Alignment Feedback System and Method, issued Nov. 1, 2022, the contents of which are incorporated herein by reference as if fully set forth herein. It also is understood that other types of instruments may be implemented and that the scope of the invention is not limited in any way by the types of instruments employed.

To provide background, a brief description of traditional spinal imaging systems will be described first. This will be followed by a general description of active instruments 12 and their use, after which, the accuracy enhancement system 10 and method will be described in detail.

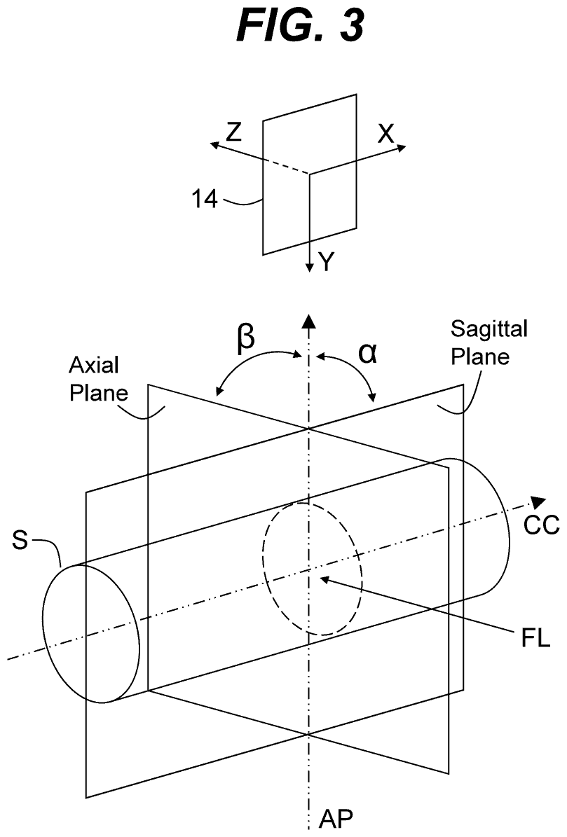

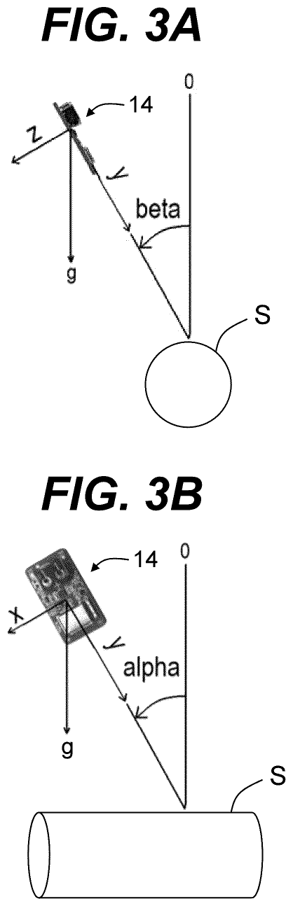

FIG. 3 shows a representation of a patient's spine S showing the sagittal plane and a general depiction of an associated sagittal angle α, and the axial plane and a general depiction of an associated axial angle β, each with respect to the patient's anteroposterior axis AP. For reference, the patient's craniocaudal axis CC also is shown as well as a representation of the alignment feedback system 14 and its associated X-axis, Y-axis and Z-axis.

Traditionally, a surgeon may utilize clinical imaging equipment, e.g., fluoroscopy, computed tomography (CT), magnetic resonance imaging (MRI), etc., to provide imaging information of a patient's spine, e.g., imagery of the patient's spine along the axial and/or sagittal plane. The imagery may be used to determine the proper placement and orientation of spinal implants such as pedicle screws (and their associated pilot holes), and other types surgical devices.

During such imaging procedures, the patient is placed onto an associated operating table and his/her anteroposterior axis AP and/or craniocaudal axis CC is aligned with the coordinate system of the imaging system (e.g., with the fluoroscope). In this way, the imaging system may provide data based on a coordinate system relative to the patient's spine (e.g., to his/her anteroposterior axis AP and/or craniocaudal axis CC).

For example, as shown in FIG. 3, a fluoroscopy system may take imaging data along the generally horizontal line-of-sight FL that is orthogonal to the patient's AP axis resulting in visual images of sagittal angles α relative to the patient's AP axis. This imaging information may be taken in real time, e.g., intraoperatively, and the surgeon may use the imagery to determine the correct sagittal angle α for a pilot hole and/or a pedicle screw based on the patient's spinal geometry and vertebral features. Notably, the fluoroscope may provide two-dimensional imagery (e.g., in the sagittal plane) but not angular information outside this plane.

Active instruments, on the other hand, may provide angular displacement data in both the sagittal plane (i.e., the sagittal angle α) and in the axial plane (i.e., the axial angle β).

In a first example, as shown in FIG. 2, the active instrument 12 includes a surgical tool or instrument 16 equipped with an alignment feedback system 14. For example, the tool 16 may be an awl or a Lenke probe used to form a pilot hole in a pedicle of a patient's spine for implantation of a pedicle screw during spinal stabilization surgery. Providing the proper position, orientation and trajectory of the tool or instrument 16 during the procedure is preferred to ensure a properly oriented pilot hole. Once the pilot hole has been formed, the pedicle screw may be implanted into the hole and driven into position. The pedicle screw preferably passes through the patient's pedicle and into the vertebral body without breaching and/or perforating the lateral or medial cortical walls.

Once the tool 16 is calibrated as described herein, and at the onset of the surgical procedure, the tool 16 is initially oriented such that the feedback system's Y-axis is aligned with the patient's AP axis and its X-axis is aligned with the patient's CC axis (as shown in FIG. 3). This also places the Y-axis of the feedback system 14 in alignment with gravity. The tip of the tool or instrument 16 may then be placed at the desired pilot hole starting point, e.g., at a known spinal reference point. The tool or instrument 16 (including the feedback system 14) may then be tilted along the axial plane and along the sagittal plane until positioned in the proper orientation for forming the pilot hole. That is, the tool 16 may be tilted at a sagittal angle α and at an axial angle β simultaneously, and preferably without rotation, or a limited amount of rotation, about the feedback system's Y-axis. The orientation of the tool 16 (e.g., the axial angle β and the sagittal angle α) is measured in real time by the feedback system 14 and the resulting measurement data is displayed and used by the surgeon to ensure the proper trajectory of the tool 16 during formation of the pilot hole. As will be described in other sections, by preferably avoiding rotation, or minimizing rotation, about the feedback system's Y-axis during this procedure, the feedback system 14 preferably makes accurate sagittal angle α and axial angle β measurements. In a preferred embodiment, the face of the feedback system 14 is maintained facing the surgeon as shown in FIG. 2. And the avoidance, or minimization, of rotation of the tool about the feedback system's Y-axis provides that the face of feedback system 14 continues to face the surgeon, as the tool 16 is tilted either along the sagittal plan to provide sagittal angle α measurements, or along the axial plane to provide axial angle β measurements. In any event, it is preferred that system 10 provides accurate measurement of composite angles, where the surgeon does not rotate the system 14 about the shaft of tool 16, or without unacceptable error being introduced from any rotation of system 14 about the shaft of tool 16.

In this example, the tool's alignment feedback system 14 may include a tri-axial accelerometer that provides simultaneous measurements of the gravitational field in three orthogonal directions (X, Y, Z), which are ultimately used to provide position, orientation and trajectory information of the system 14 for the surgeon. However, electronic accelerometers are generally not perfect measuring devices, and as such, the alignment feedback system 14, containing the accelerometer(s), preferably undergoes a calibration and/or optimization procedure prior to use, so that the alignment feedback system 14 may provide accurate measurement results during later surgical procedures.

As described herein, the system 10 provides a method that accounts for the performance deficiencies and/or the limitations of the alignment feedback system 14 (e.g., its accelerometers) as they pertain to the specific use case of the feedback system 14 and its associated tool 16. In addition, the repeatability of the accelerometers may be measured, and the overall measurement uncertainty also may be calculated.

The accelerometers within the alignment feedback system 14 provide data based on its chip-based coordinate system and relative to gravity (e.g., to a vertical plumb line). Because of this, the patient's anteroposterior axis AP and the associated imaging system (e.g., the fluoroscope) are preferably aligned with gravity by adjusting the table on which the patient lays. The X-axis, Y-axis and Z-axis of the feedback system 14 is then aligned with the patient's anteroposterior axis AP and craniocaudal axis CC as shown in FIG. 3. Once in this position, the tool 16 may be tilted at an angle β in the axial plane and at an angle α in the sagittal plane, simultaneously, and preferably without rotation, or minimal or reduced rotation, about the feedback system's Y-axis (so that the face of the alignment feedback system 14 remains facing the surgeon). In this way, the feedback system 14 provides accurate sagittal a and axial β tilt information with respect to its Y-axis, gravity, and the patient's AP axis (preferably all aligned).

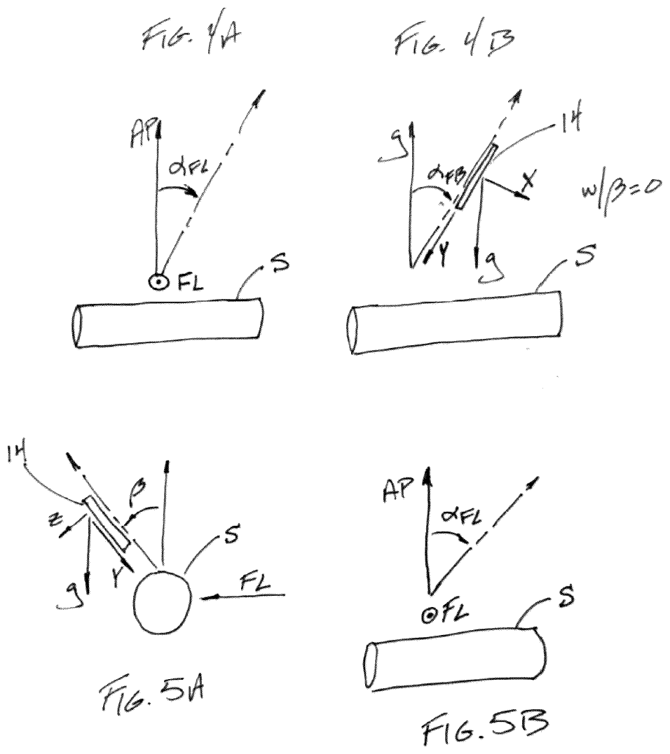

Using FIG. 3 as a reference, FIG. 4A shows a sagittal angle α_FL measured by the fluoroscope from the perspective of FL (into the page) and FIG. 4B shows a sagittal angle α_FB measured by the feedback system 14 from the perspective of its Y-axis and with no tilt in the axial plane (i.e., the axial angle β=0). As shown, in this arrangement α_FL=α_FB and the angular data from the two systems match.

However, when the feedback system 14 is also tilted in the axial plane (away from the fluoroscope's line-of-sight FL as shown in FIG. 5A), the sagittal angle α_FL that the fluoroscope is attempting to measure from the perspective of FL becomes compound, i.e., it becomes a vectorial combination of the angles α, β, and shifts from the depiction in FIG. 4A to the depiction in FIG. 5B (it increases). In this case, the measured sagittal angle α_FL of FIG. 5B is a vectoral combination of the sagittal angle α_FL of FIG. 4A and the axial angle β of FIG. 5A projected onto the 2-dimensional sagittal plane as seen by the fluoroscope from the perspective of FL (shown in FIG. 3). Notably, the sagittal angle α_FB measured by the feedback system 14 may preferably remain constant and independent of the axial angle β as long as the feedback system 14 has not been rotated about its Y-axis. The reason for this will be described in other sections.

This phenomenon may be referred to as foreshortening. As such, when the axial angle β≠0, the sagittal angle α_FB read by the feedback system 14 may not match the sagittal angle α_FL as seen by the fluoroscope. To account for this, the system 10 preferably correlates the data provided by the active instrument 12 with the data provided by the other traditional imaging systems (e.g., the fluoroscope) such that the sagittal angle α as measured by both systems may be shown to match. As such, the angles measured by the feedback system 14 may be verified by an independent method (i.e., by the fluoroscope) thereby providing confidence that the axial and sagittal angles β, a measured by the feedback system 14 may be accurate. This will be described in other sections.

### Calibration of the System 10

In some embodiments, the system 10 provides instrumentation 100 and methodologies (e.g., calibration fixtures and procedures) to enhance the accuracy of the active instrument 12, and in this case, the accuracy of the tool or instrument 16 equipped with the alignment feedback system 14.

Accelerometers are known to have performance deficiencies caused by gain errors, offset errors, channel crosstalk, and other errors during operation. Because the three axes of the tri-axial accelerometer used by the alignment feedback system 14 are independent of one another, maximum (+g) and minimum (−g) measurements made along each of the X-, Y-, and Z-axis may not match (resulting in gain errors) and may show non-zero values at zero (resulting in offset errors). Other types of errors also may be quantified and corrected for as described herein.

The accelerometer chip may be initially calibrated by the manufacturer, but because this manufacturer calibration may not account for the particular use case of the chip (i.e., for simultaneously measuring sagittal and axial angles α, β), this may not sufficiently address the foregoing errors. Also, there may be errors induced during assembly of the chip or board comprising system 14, such as errors that may be caused by the mechanical stress of soldering or from board-to-board variations in the physical placement of the accelerometer on the board.

As such, the accelerometer chip or system 14 is preferably recalibrated or further calibrated during or after manufacturing the sensor 14 to ensure or enhance its accuracy during later surgical procedures. It is preferred that the calibration process may also test the performance of system 14 over its operating angular range.

In general, calibration of the system 14 improves its accuracy in the angular feedback it provides during later surgical procedures, especially in situations where both the sagittal angle and axial angle are displaced from vertical by an increasing amount, i.e., to improve angular feedback for such composite angles. As discussed herein, it is desired that the surgeon not rotate the system 14 about the axis of the tool 16, or to minimize any such rotation, because such rotation may introduce error into the angular feedback provided. However, by calibrating the system 14 prior to any use in surgery, the error that might otherwise exist because of the accelerometer's inherent limitations, may be reduced or eliminated. This in turn helps reduce the overall, aggregate error of the system 14 that may include some amount of error introduced by a surgeon rotating the system 14 about the tool's axis. As such, the calibration methods and systems of the current invention may address error introduced by a surgeon's rotating the tool 16 because the overall error of the accelerometer and rotation may still be within pertinent regulations.

The overall calibration of the current invention may include two main calibration procedures. A first calibration procedure may include calibrating the accelerometer chip or system 14 for quantifiable errors in the X-, Y-, and Z-planes. This first calibration may result in a first set of correction factors that may be applied to raw X, Y, and Z data taken by the system 14 during use to provide corrected X, Y, and Z data. As will be described herein, this corrected X, Y, and Z data may then be used by the system 14 to determine initial sagittal and axial angles α, β. However, these initial sagittal and axial angles α, β may include additional errors, e.g., errors associated with the system's operational response ranges for α and β. Accordingly, a second calibration procedure may include calibrating the system 14 for errors in the operational response ranges for α and β resulting in a set of correction factors that may be applied to the initial sagittal and axial angles α, β to provide corrected sagittal and axial angles α, β data. In a preferred embodiment, the sensor 14 may perform the computations involved in both calibration procedures, and also may determine whether a particular sensor 14 passes or fails the calibration.

In some embodiments, a calibrator, which may include a calibration text fixture attached to a two- or three-axis gimbal, may be used for both or either the first and second calibration procedures. For example, during the first calibration procedure, the gimbal may position the test fixture, and thus the sensor(s) 14, at various angles in X, Y, and Z to determine the X, Y, and Z correction factors. These X, Y, and Z correction factors may then be used to determine the initial sagittal and axial angles α, β. Then, during the second calibration procedure, the gimbal may position the sensor(s) 14 at various sagittal and axial angles α, β to determine the α and β correction factors. After the second calibration procedure, the α and β correction factors may be used to provide corrected sagittal and axial angles α, β data.

In either or both of the first and second calibration procedures, the X, Y, Z and/or the α and β correction factors may be computed and preferably stored in the accelerometer, e.g., stored in FLASH memory of the processor chip (on which the accelerometer resides). The gimbal axes may be controlled by stepper motors, which are in turn controlled by a test controller. Prior to using the test fixture, the gimbal axes are preferably aligned to gravity with the aid of an NIST-traceable digital protractor. The test controller, which may comprise a PCB, may position the fixture and sensor(s) 14 at proper angles each step of the calibration process and to communicate to the sensor(s) 14 when each angle has been reached.

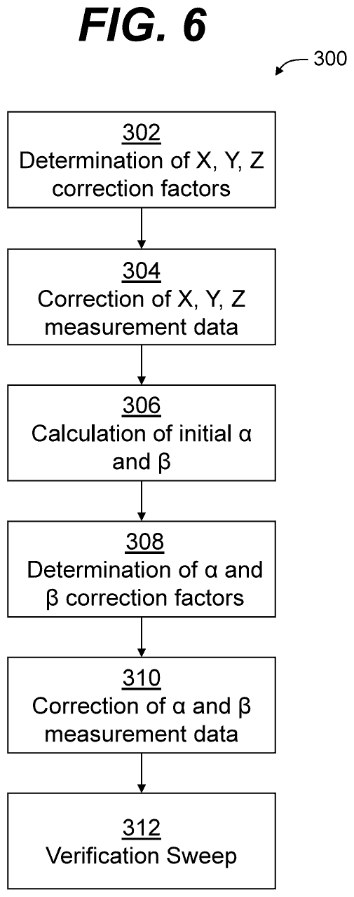

Details of the overall calibration procedure 300 are described below with reference to FIGS. 6-10, and with steps or actions 302-304 pertaining primarily to the first calibration procedure (i.e., the X, Y, and Z calibration described above) and with steps 308-310 pertaining primarily to the second calibration procedure (i.e., the α and β calibration described above).

In some embodiments, as shown in FIG. 6, the system 10 may include software and hardware which determines and corrects for the associated errors by performing the following steps, actions, or procedure 300. The calibration methods discussed below also may occur in a separate calibration system where alignment feedback systems or measurement sensors 14 may be loaded into instrumentation 100 or other calibration equipment that may be separate and apart from the system 10 shown in FIG. 1.

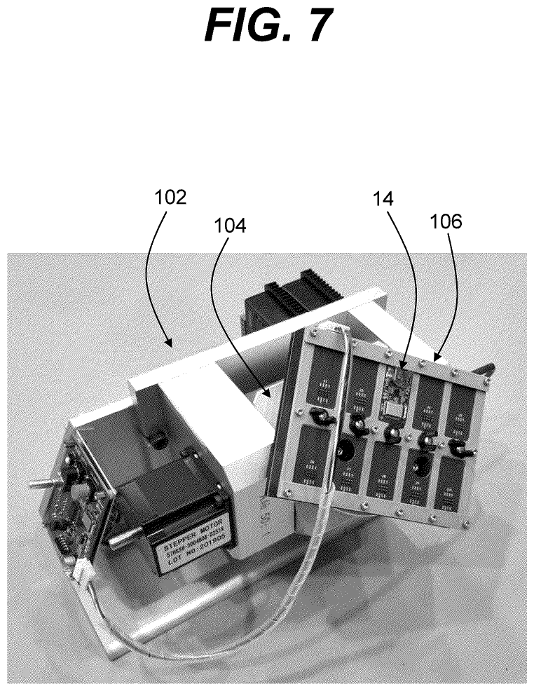

302: Determine X, Y, Z correction factors, for example, through use of a calibration fixture 102 (FIG. 7);

304: Correct raw X, Y and Z measurement data taken by the system 14 using the X, Y, Z correction factors from 302 to result in corrected X, Y, Z measurement data;

306: Use the X, Y, Z corrected measurement data from 304 to calculate the initial sagittal angles α and/or the initial axial angles β;

308: Determine α and β response correction factors, for example, through use of a calibration fixture 102 (FIG. 7);

310: Correct the initial sagittal angles α and/or the initial axial angles β from 306 using the α and β response correction factors from 308 to result in corrected α and β measurement data;

312: Perform a two-dimensional verification sweep.

To determine the X, Y, Z correction factors in 302, the system 10 may include a positioning calibration device 102 (preferably traceable to national standards) that includes a gimbal 104 (and/or other type(s) of suitable positioning mechanisms) configured to physically position a fixture 106 at various orientations (see FIG. 7). The alignment feedback system 14 may be loaded onto the fixture 106, and the system 10 may cause the calibration device 102 to position the fixture 106 and the feedback system 14 (via the gimbal 104) at orientations chosen specifically to each cause a full-scale reading on one axis (e.g., the Y-axis) of the feedback system 14 while causing a near-zero reading on the other two axes (e.g., the X-, and Z-axis). During this process, readings from the feedback system 14 are taken at each position. The measured values are then compared to theoretical ideal values and the X, Y, Z correction factors are determined. The correction factors may be calculated by the feedback system 14, by the calibration fixture 102, by the system 10, by an associated controller, and/or by any combinations thereof. The fixture 106 may include multiple slots such that multiple feedback systems 14 may be calibrated simultaneously, e.g., FIG. 7 shows ten available slots with one slot populated by a single feedback system 14.

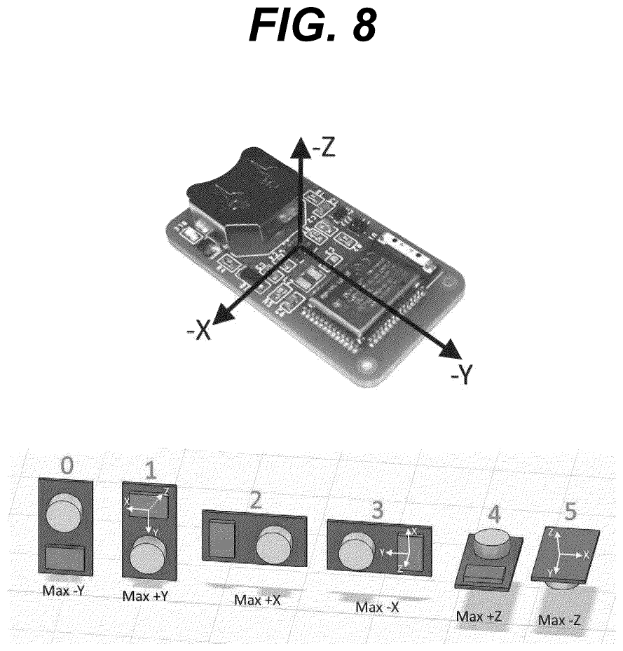

For example, in some embodiments, the alignment feedback system 14 is systematically positioned in each of the orientations 0-5 as shown in FIG. 8, and readings are taken from the feedback system 14 at each position. As shown, positions 0 and 1 position the feedback system's Y-axis at maximum and minimum orientations, respectively, while setting the feedback system's X- and Z-axes perpendicular to gravity (and therefore theoretically at zero). Similarly, positions 2 and 3 set the feedback system's X-axis at maximum and minimum orientations, respectively, while setting the Y- and Z-axes at zero, and positions 4 and 5 set the feedback system's Z-axis at maximum and minimum orientations, respectively, while setting the X- and Y-axes at zero. For reference, FIG. 8 also shows the default orientation of the alignment feedback system 14 with respect to its X-, Y-, and Z-axes.

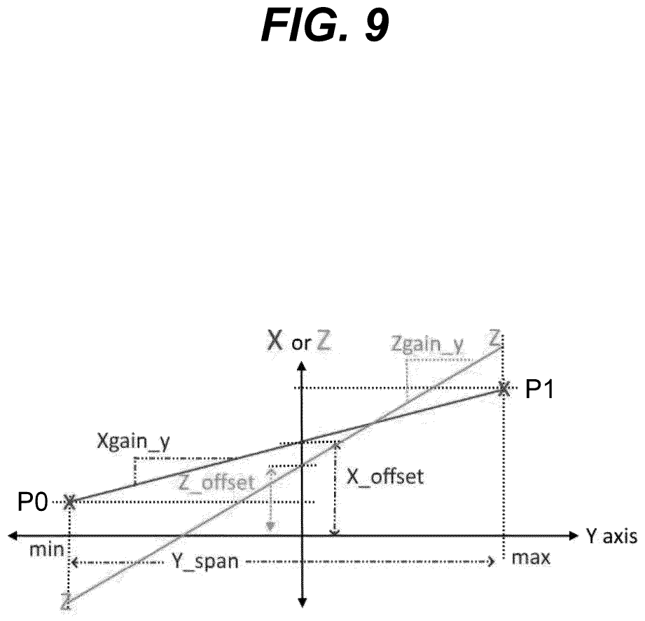

FIG. 9 shows a graphical representation of the feedback system's response when first oriented in position 0 (P0) and then moved to position 1 (P1). As shown, because the feedback system 14 is nonideal, the absolute value of the minimum P0 and maximum P1 values in Y are typically not equal and thus the Y-span is not symmetrical about the zero point. In addition, the values in X and in Z vary from an ideal response of zero (being perpendicular to gravity) and instead show variations with Y caused by static offset errors (e.g., X_offset and Z_offset in FIG. 9). Similar errors occur in X and Z when the feedback system 14 is oriented in positions 2 and 3, and in 4 and 5, respectively.

In some embodiments, the above procedure results in a quantification of a total of twelve static offset errors, four for each of the X-axis, Y-axis, and Z-axis. Given that each of the four static offset errors for each axis may carry the same weight, the offset errors for each axis may be averaged resulting in a single static offset error for each.

In addition, in some embodiments, because the slopes connecting the values in X and in Z in FIG. 9 are non-zero, a dynamic offset error also may exist as a function of these axes. This may include cross talk between one axis (e.g., the Y-axis) and the other two axes (e.g., the X-axis and Z-axis) and may be expressed as gain, e.g., as Xgain_y (the offset in X due to the Y-axis value) and Zgain_y (the offset in Z due to the Y-axis value) as shown.

In some embodiments, the system 10 determines the total offset error correction factors for each of the X-axis, Y-axis, and Z-axis by combining the respective static offset errors with the respective dynamic offset errors. For example, the system 10 may determine the total offset error correction factors as shown below.

$$X\_offset = Xstatic\_offset + ( Ystatic\_offset * Xgain\_y ) + ( Zstatic\_offset * Xgain\_z )$$

$$Y\_offset = Ystatic\_offset + ( Xstatic\_offset * Ygain\_x ) + ( Zstatic\_offset * Ygain\_z )$$

$$Z\_offset = Zstatic\_offset + ( Xstatic\_offset * Zgain\_x ) + ( Ystatic\_offset * Zgain\_y )$$

For reference, Table 1 below shows the systematic error correction factors associated with the alignment feedback system 14 that the system 10 may determine in step or action 302. Other errors also may exist and may be accounted for.

TABLE 1
Systematic Error Correction Factors
Associated with the Alignment Feedback System

| | |
|---|---|
| X_offset | The average value of the X-axis when it is perpendicular to gravity and |
| | either the Y-axis or Z-axis is at a maximum or minimum placement; |
| Y_offset | The average value of the Y-axis when it is perpendicular to gravity and |
| | either the X-axis or Z-axis is at a maximum or minimum placement; |
| Z_offset | The average value of the Z-axis when it is perpendicular to gravity and |
| | either the X-axis or Y-axis is at a maximum or minimum placement; |
| X_span | The difference between the X-axis maximum and X-axis minimum values; |
| Y_span | The difference between the Y-axis maximum and Y-axis minimum values; |
| Z_span | The difference between the Z-axis maximum and Z-axis minimum values; |
| Xgain_y | The variation in X-axis measurements when X is held constant and Y |
| | varies from maximum to minimum; |
| Xgain_z | The variation in X-axis measurements when X is held constant and Z |
| | varies from maximum to minimum; |
| Ygain_x | The variation in Y-axis measurements when Y is held constant and X |
| | varies from maximum to minimum; |
| Ygain_z | The variation in Y-axis measurements when Y is held constant and Z |
| | varies from maximum to minimum; |
| Zgain_x | The variation in Z-axis measurements when Z is held constant and X |
| | varies from maximum to minimum; and |
| Zgain_y | The variation in Z-axis measurements when Z is held constant and Y |
| | varies from maximum to minimum. |

To perform a correction of X, Y, Z, as in step or action 304, the system 10 applies the determined error correction factors to the raw X, Y and Z measurement data resulting in corrected measurement data as shown below.

First, the offset error correction factors are subtracted from the raw measurement data to produce offset-adjusted data as follows:

$${Xo}_{1} = Xraw - X\_offset$$

$${Yo}_{1} = Yraw - Y\_offset$$

$${Zo}_{1} = Zraw - Z\_offset$$

Second, the cross-channel offset correction factors are subtracted out:

$$X {o}_{2} = {Xo}_{1} - Zo / Xgain\_z - Yo / Xgain\_y$$

$${Yo}_{2} = {Yo}_{1} - Xo / Ygain\_x - Zo / Ygain\_z$$

$${Zo}_{2} = {Zo}_{1} - Xo / Zgain\_x - Yo / Zgain\_y$$

And third, the X and Z values are normalized to the span of the Y axis:

$$Xn = {Xo}_{2} * ( Y\_span / X\_span )$$

$$Zn = {Zo}_{2} * ( Y\_span / Z\_span )$$

This process results in corrected X, Y, and Z measurement data with the resulting offset errors approaching zero and with optimized spans for each of the X-, Y-, and Z-axis that are generally equal.

The initial α and β angles are determined in step or action 306 using the trigonometric relationships between the corrected X, Y, and Z measurement data as shown below.

$$α = arctan ( X / Y )$$

where X and Y are the corrected values from correction 304; and

$$β = arcsin ( Z / g )$$

where Z is the corrected value from correction 304 and g is the maximum value of Z taken from the Z_span value determined in error coefficient determination 302.

As shown above, the axial angle β is a function of the Z value and gravity and is independent of the X and/or Y values. As such, the Z-axis (which is orthogonal to the feedback system's PCB) may register a constant axial angle β independent of any sagittal tilt angle α (as long as rotation of the alignment feedback system 14 about its Y-axis during its use is avoided or minimized as described above). Put another way, by preferably avoiding rotation about the Y-axis, tilting the alignment feedback system 14 in the sagittal plane is generally equivalent to simply rotating the feedback system 14 about its Z-axis while keeping the Z-axis constant. In doing so, the values of X and Y will change, but the value of Z will remain constant. Accordingly, any gain and/or offset errors in the X- and/or Y-axis channels of the feedback system 14 preferably do not adversely affect the axial angle β calculation.

The current invention preferably separates the sagittal angle calculation based on X and Y, from the axial angle calculation based solely on Z.

The axial angle β also may be calculated as the arctan (Z/Y), but in this case, when the deviation of α increases, the denominator (Y) decreases thereby causing this calculation to be subject to error. Using the arcsin (Z/g) instead, the denominator (gravity g) remains constant, and the angle registered in the Z-axis is not a function of the α orientation of the alignment feedback system 14 (i.e., the axial angle β is not a function of X or Y).

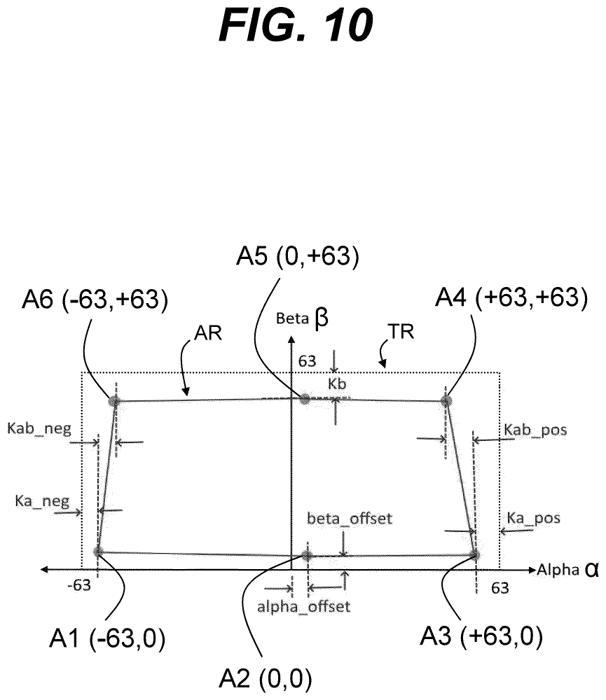

To perform a correction of the initial α and β data determined in 306, as in step or action 308, the system 10 may again use a calibration fixture 102. FIG. 10 shows the theoretical operational range TR of the alignment feedback system 14 represented by the outer dashed bounding lines. In some embodiments, this range TR is about −63° to +63° for the sagittal angle α and about 0° to +63° for the axial angle β. The actual response of the feedback system 14 typically differs from this theoretical response TR, and as such, the system 10 may account for this difference.

To do so, the alignment feedback system 14 is loaded onto the positioning calibration fixture 102 and the fixture 102 is controlled to position the feedback system 14 at the angular extremes (α, β)=(−63,0), (0,0), (+63,0), (+63,+63), (0,+63), and (−63,+63). Example measurement results are plotted as A1-A6 in FIG. 10 and the resulting actual response AR is represented as the solid inner bounding lines extending between these points A1-A6. Error correction factors accounting for the difference between the theoretical operational response TR and the actual measured response AR are then determined. These error correction factors are shown below in Table 2.

TABLE 2
Systematic Error Correction Factors
Associated with the Alignment Feedback System

| | |
|---|---|
| alpha_offset | The measured value of α when |
| | α is set to zero; |
| beta_offset | The measured value of β when |
| | β is set to zero; |
| Ka_pos | The amount α varies with α for |
| | positive values of α; |
| Ka_neg | The amount α varies with α for |
| | negative values of α; |
| Kab_pos | The amount α varies with α and β for |
| | positive values of α; |
| Kab_neg | The amount α varies with α and β for |
| | negative values of α; |
| Kb | The amount β varies with β for |
| | positive values of β; |

Notably, Kb does not exist for negative values of β as β is always positive. The K-factors shown above may be defined as the inverse of the equivalent gains, and as such, may include numerically large integers. In this way, application of the corrections using mathematical division is preferably less prone to errors.

These correction factors may be applied to the initial measured value of a as shown below, allowing for positive/negative asymmetry.

$$α = raw\_alpha - alpha\_offset - raw\_alpha / Ka\_pos - ( raw\_alpha * raw\_beta ) / Kab\_pos ;$$

$$α = raw\_alpha - alpha\_offset - raw\_alpha / Ka\_neg - ( raw\_alpha * raw\_beta ) / Kab\_neg ;$$

where α is the corrected value of α;
raw_alpha is the initial measured value of α; and
raw_beta is the initial measured valued of β.

The corrections may be applied to the initial measured value of β as shown below:

$$β = raw\_beta - beta\_offset - raw\_beta / Kb\_pos ;$$

where β is the corrected value of β; and
raw_beta is the initial measured valued of β.

The above corrections may result in corrected β values typically within about 1° of true value and corrected α values within about ±0.5° of true value. In addition, the resulting error distribution is approximately sinusoidal, with a peak error occurring at roughly 45° from vertical and tapering off to zero at vertical and the ±63° deviation limit.

In some embodiments, the system 10 may determine a final set of correction factors by causing the positioning calibration fixture 102 to sweep the alignment feedback system 14 in two dimensions over its entire operating range of 0° to 63° degrees in β, and −63° to +63° in a, in 4.5° increments (or similar). At each position, the alignment feedback system 14 (and/or the system 10) may compare its computed α and β values (as determined in steps or actions 302-308) to the true values of the positioning calibration fixture 102, and the differences may be stored in memory as final correction factors (e.g., in a lookup table within the feedback system 14, within the system 10, within the controller 200, etc.). Then, during use, the alignment feedback system 14 (and/or the system 10) may look up the appropriate correction factor for each α and β measurement reading and apply the correction factor(s) to provide corrected measurement data. In some embodiments, it may be preferable to have sufficient granularity of data within the correction factor tables such that interpolation is not required. However, data interpolation and/or extrapolation is also within the scope of the current invention.

For example, in some embodiments, a lookup table of correction factors may be indexed by the axial angle β measurement values (since these values may be independent of the raw X and Y values as describe above) such that the associated sagittal angle α correction factors may be thereby referenced.

To perform a verification sweep, as in step or action 312, in some embodiments, a verification sweep of the alignment feedback system 14 may be performed over its full operating range (or portion(s) thereof) to compare the final corrected values of α and β to the true values of the calibration fixture 102. Differences between the two may be stored in memory and may be regarded as measurement errors generally defining the accuracy of the alignment feedback system 14. In some embodiments, if the measurement errors exceed a predefined threshold (e.g., ±0.3°), the feedback system 14 may be deemed to have failed the calibration procedure and its use may be restricted by the system 10. The pass/fail determination may be based on the largest error value found. The pass/fail determination may be indicated using the on-board LEDs and may also be recorded in the feedback system 14. As a further precaution, any sensor or feedback system 14 that fails calibration may be inoperable and/or will not communicate with the display that would otherwise be viewed by the surgeon.

It is understood that not all of the actions 300 are required to be taken; additional steps or actions may also be taken, and the actions 300 may be taken in different orders.

Performing calibration preferably results in enhanced accuracy of sagittal and axial angular measurements that are displayed to the surgeon. For example, system 10 may provide sagittal and axial angular measurements with +/−1 degree system-level accuracy, i.e., which may be defined as the accuracy of the system 10 as used in an idealized setting. As another example, system 10 may perform at +/−3 degree procedural accuracy.

Correlation with Imaging Instrumentation

As described in other sections, when the active instrument 12 (which includes the alignment feedback system 14) is tilted in both the sagittal plane at a sagittal angle α and in the axial plane at an axial angle β, the angular data measured by the feedback system 14 may not match corresponding angular data taken by a fluoroscope from a line-of-sight orthogonal to the patient's anteroposterior axis AP (see FIGS. 3, 4A, 4B, 5A, and 5B). In some embodiments, the system 10 is designed to account for this by correlating the data provided by the alignment feedback system 14 with the data provided by the fluoroscope. In some embodiments, this is accomplished by foreshortening a sagittal angle α_FB measured by the feedback system 14 to correlate with a sagittal angle α_FL measured by the fluoroscope.

In some embodiments, the system 10 first reads (or otherwise receives) the sagittal angle α_FB and the axial angle β measured by the alignment feedback system 14. The system 10 may then foreshorten the feedback system's sagittal angle α_FB as shown below.

$$α\_FS = arctan ( tan ( α\_FB ) / cos ( β ) )$$

where α_FS=the foreshortened sagittal angle;
   α_FB=the sagittal angle measured by the alignment feedback system 14; and
   β=the axial angle measured by the alignment feedback system 14.

In some embodiments, the system 10 may then compare the foreshortened sagittal angle α_FS with the fluoroscope's measured sagittal angle α_FL. If the system 10 determines that the foreshortened sagittal angle α_FS substantially matches the fluoroscope's measured sagittal angle α_FL within a predetermined threshold value (e.g., within the measurement uncertainty), the system 10 may deem that the angular measurements taken by the alignment feedback system 14 are accurately correlated with the angular measurements taken by the fluoroscope. That is, the angular measurements taken by the alignment feedback system 14 have been verified using an alternate and independent method (i.e., using the fluoroscope system).

In this case, the system 10 may enable further use of the feedback system 14 (e.g., by enabling use of the system 14 through its associated mobile application). However, if the resulting foreshortened sagittal angle α_FS does not substantially match the fluoroscope's measured sagittal angle α_FL within the measurement uncertainty, the system 10 may deem that the systems do not correlate and the system 10 may restrict the use of the active instrument 14 (e.g., by disabling use of the system 14 through its associated mobile application).

For purposes of this specification, the term “substantially matches” may generally mean “matches within a predetermined level of uncertainty”.

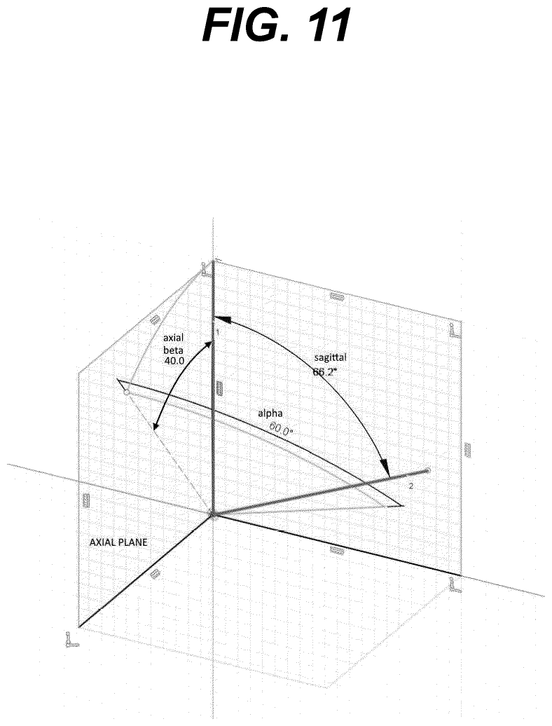

An example of the above process is shown in FIG. 11 where α_FB=60° and β=40°, resulting in a foreshortened sagittal angle α_FS=66°. In this example, if the sagittal angle α_FL measured by the fluoroscope also equals 66° such that α_FL=α_FS, the system 10 may deem that the systems are correlated and may enable use of the feedback system 14 to take accurate measurements.

It is appreciated that the system 10 may correlate any compound angular data taken by the alignment feedback system 14 with any other angular data taken by separate imaging systems (e.g., fluoroscopes) by foreshortening the data using the same or similar methodologies as described above. As such, the scope of the current invention and operation of the system 10 is not limited in any way by the angular data and/or by the source(s) of the angular data that it may be used to correlate.

### Active Instrument Guide Member 20; Handling of the Alignment Feedback System

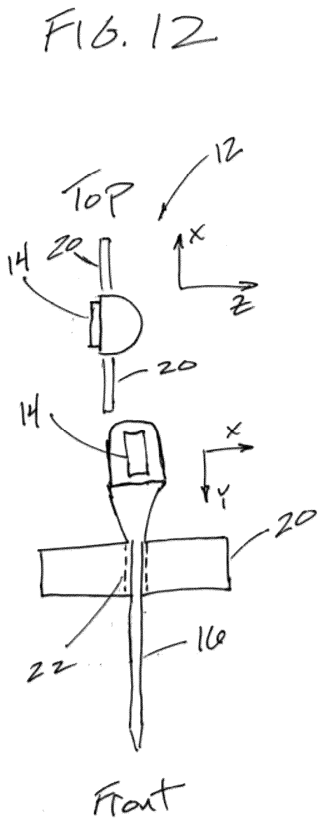

In some embodiments, as shown in FIG. 12, the system 10 may provide a guide member 20 to the active instrument 12. More specifically, the system 10 may provide the guide member 20 for use with the alignment feedback system 14, e.g., on the tool or instrument 16, to assist the surgeon in keeping the feedback system 14 properly rotationally oriented for accurate sagittal angle α and axial angle β tilt measurements.

As described in other sections, prior to using the tool 16, the surgeon first aligns the patient's anteroposterior axis AP with gravity, and then align the X-axis, Y-axis and Z-axis of the feedback system 14 with the patient's anteroposterior axis AP and craniocaudal axis CC (see FIG. 3). Once in this position, the tool 16 may be tilted while taking sagittal angle α and axial angle β tilt readings, but is preferably not rotated about its Y-axis (as this type of rotation may invalidate or affect the accuracy of the feedback system's tilt readings).

To help guide the surgeon with this technique, and/or for training purposes, the system 10 may include a guide member 20 configured as a visual indicator. In some embodiments, the guide member 20 includes an elongate plate or other suitably formed structure that attaches to the tool 16 (e.g., right below the handle) and that extends away from the tool 16 on one and/or both sides along its X-axis. In this way, the guide member 20 provides a visual representation of the feedback system's X-axis that is easy to view and understand. When the tool 16 is used, the surgeon may tilt the tool 16 at the desired sagittal and axial angles α, β while keeping the guide member 20 aligned with the feedback system's X-axis to measure the angles α, β with the feedback system 14. Once the orientation of the tool 16 is confirmed (using the readings from above), the surgeon may then manipulate the tool 16 (e.g., rotate it) to bore the pedicle screw pilot hole. When the surgeon wishes to check the tool's angular orientation information again, he/she may view the guide member 20, rotate the tool 16 as needed to align the guide member 20 with the patient's craniocaudal axis CC, and then take the angular readings. In this way, the tool's X-axis is rotationally realigned properly and any rotation of the tool 16 that occurred when forming the pilot hole may be removed during the tilt information measurement.

In some embodiments, the guide member 20 may include a visible light line emitter (e.g., a laser) that emits a straight line of visible light in the forward and/or reverse directions along the tool's X-axis. The visible line of light may be used by the surgeon to visually align the tool 16 when taking angular readings as described above.

In some embodiments, the guide member 20 is releasably attached to the tool 16 using a releasable attachment mechanism 22 (e.g., a clamp or other suitable mechanism) so that it may be easily removed and/or attached as needed. For example, the guide member 20 may be attached to the tool 16 for training purposes and subsequently removed when deemed to be no longer needed.

In some embodiments, the attachment mechanism 22 includes guide structures (e.g., slots, pins, etc.) that ensure the correct orientation of the guide member 20 along the feedback system's X-axis each time it is attached.

In other embodiments, the face of the alignment system 14 may be facing the surgeon when tool is properly held by the surgeon, and the system is preferably not rotated about the axis of its shaft, or its rotation is limited. In these embodiments, the surgeon may manipulate the tool 16 so that the face of the sensor or system 14 remains facing him or her during the portions of the procedure when angular feedback is desired. In other words, the surgeon may tilt the system 14 in the sagittal and/or axial plane(s) while preferably avoiding or minimizing rotation of the shaft of the tool 16, so that the face of the system 14 remains facing him or her. The face of the system 14 may be a different color than the handle or tool it is attached to so as to facilitate the surgeon's awareness of whether the system 14 is facing the surgeon.

The manipulation of certain handheld surgical instruments may require rotation about its axis, e.g., a screwdriver's rotation, or rotation of a probe in order to form a pilot hole. While such rotation may affect the sagittal and axial angles displayed to the surgeon, the surgeon may still bring the instrument back to a position so that the sensor or system 14 is facing him or her when angular measurement feedback is obtained, to ensure that the axis of the instrument is still oriented in the desired trajectory.

It has been found that surgeon's may exhibit sufficient control over the tool 16 and the alignment feedback system 14 attached to it, so as to avoid rotation about the tool's shaft, or to minimize or reduce rotation so that the resulting angular measurement feedback is sufficiently accurate to meet pertinent standards. In other words, tests have shown that the proprioception of spinal surgeons was sufficient to display sufficient clinical accuracy of alignment system 14. For example, it has been seen that surgeon's may consistently avoid rotation or maintain rotation at about or below about 5 degrees rotation which preferably results in aggregate axial and sagittal angular measurement feedback within pertinent standards, e.g., 3 degree aggregate error limit.

As mentioned above, it is preferred that the chips or systems 14 are calibrated prior to their use in surgery. This preferably reduces error of the system 14 overall, and may address some error that may be introduced by surgeon rotation, if indeed the system 14 is rotated at all.

It is appreciated that while the above describes the guide member 20 as being configured with the tool 16 beneath its handle, it is understood that the guide member 20 may be configured at any suitable location on the tool 16 and/or directly with the alignment feedback system 14.

### Power Supply and Duration of Power to the Alignment System

Another aspect of the current invention, regarding the power supply of the alignment feedback system 14, and the duration the power supply, is now described. As set forth in U.S. Pat. No. 11,484,381, entitled Instrument Alignment Feedback System and Method, incorporated by reference herein above, the alignment feedback system 14 may include a power supply such as a battery. For example, a 1.5 volt coin cell may be used to power feedback system 14. As also described in the '381 patent, certain embodiments of the alignment feedback system 14 may be disposable and designed to be used once and then discarded.

The alignment feedback system 14 may be programmed, may contain software and/or firmware, or may otherwise provide instruction that limits or controls the duration of power being supplied. This may be preferred where, for example, the alignment feedback system 14 is intended for single-use operation, or to be disposable. The system 14 may be initially provided to the surgeon, hospital or other medical facility, in a sterile condition. And after the single use, the system 14 may be discarded, thereby avoiding any concern that a non-sterile system 14 is used in a subsequent medical procedure. Indeed, the alignment feedback system 14 may generally comprise or include an integrated system of components on a printed circuit board (PCB) or chip that would not withstand the temperatures of sterilization.

The power supply, such as a battery, may be located on a PCB that contains other components of system 14. The system 14 may be designed for a predetermined or limited duration of operation. To this end, the system 14 may include a polyimide pull-tab inserted underneath the battery, such that when it is removed, the system 14 is turned on, and is able to be paired with a tablet or other display device. At this point, the battery may start powering the system 14, and the life of the system 14 may begin. As described below, the system 14 may have controls over how long the system 14 may operate.

In one embodiment, system 14 may include a timer that may stop operation of system 14 at a prescribed time, e.g., ten (10) hours, after the battery was activated. In this embodiment, the battery may still have life after the system 14 has been deactivated, but the deactivation of the system 14 still stops its operation. The elapsed time of operation may be continuously updated in a non-volatile memory included in the system 14. The prescribed time may vary, and may be set for an anticipated time a surgery may take, so as to provide for a single-use, followed by disposal.

Where the system 14 involves the angle computations described above, the system 14 may consume more power, thereby reducing battery life to below the prescribed time. In this situation, the possibility may exist that until the prescribed time is reached, the battery could be replaced so as to prolong the use of the system 14 over multiple surgeries over an extended time period by removing and replacing the battery when not actively in use. This would be contrary to the system 14 operating in a single-use, disposable manner.

Another embodiment may address this situation by preventing a battery replacement from reactivating the system 14, thereby enforcing the prescribed run time limit. This may be accomplished by setting a flag in non-volatile memory (FLASH) after the system 14 has continuously run for a prescribed amount of time, e.g., 15 minutes. The flag may be examined upon any power-up, i.e., when the original pull tab is removed or if the battery is removed and replaced. Should the flag be set, the system 14 may erase its FLASH memory rendering it inoperative. The prescribed time, e.g., the 15-minute delay, may provide time to test the system 14 and remove and reinstall the battery multiple times as needed without setting the flag, as long as the system 14 does not run for more than the prescribed time. Should someone wish to try to extend the life of the system 14 by removing the battery after a surgery (where the surgery takes longer than the prescribed time), reinstalling the battery for a second surgery would preferably erase the memory.

### Software

Software or programming that may be incorporated into and/or used with system 10 is now further described. In the following description the term “RJB” may generally refer to system 10.

### General Software Description

The programmable software that comprises the RJB system 10 may take two forms: 1) programmed firmware loaded onto the integrated chip on the RJB device itself, and 2) the software of the RJB Application (App) run on a tablet. A description of each type of software is presented below.

The core functionality of the RJB device may be driven by the programmed firmware. It is responsible for data collection, manipulation, formatting, and output via Bluetooth to the app for display to the user.

In an embodiment, the RJB App need not perform any data manipulation or calculation functions once the data stream is received from the RJB device; it is solely display software.

### Firmware

The programmed firmware allows the RJB hardware to measure angular displacement from vertical by using a Micro Electro-Mechanical System (MEMS) accelerometer to measure the gravitational field in all three axes. The three gravitational measurements are converted into angles by basic trigonometry. The displacement angles may then be communicated to a display device (e.g., RJB App run on a tablet) by a Bluetooth Low Energy (BLE) wireless link or other wireless protocol.

The MEMS accelerometer uses a spring-supported mass which deflects due to gravity. The displacement is measured through the change in capacitance between plates attached to the mass and stationary plates attached to the substrate. The change in capacitance is converted into units of micro-Gs by circuitry onchip. By using three separate structures, orientated orthogonally, the gravitational field can be measured in all three axes, x, y, and z. The accelerometer used in the RJB is preferably a 3-axis device with 14-bit resolution over a +/−2 g range.

An integrated microprocessor and RF radio module are used to control the accelerometer, compute the angles, and send the results to the display device.

### App Software

The RJB App is the tablet application for the RJB device, using Bluetooth 4.0 to connect to the device. The RJB hardware outputs the Axial and Sagittal angles through its Bluetooth connection to the tablet, which then displays the angles of alignment via the App. Written with Swift, the RJB App is available on both IOS and Android platforms.

The user connects the RJB devices to the RJB App upon startup of the application. If no devices are paired, the user will be taken to a welcome screen that contains a text box for device serial number input. Once paired, the user can rename or remove the paired device depending on need. Up to four total RJB devices can be paired at once, with the option to rename or remove each device. After pairing, the user can choose which device to have selected when the selected device data is communicated to the application. The RJB application will display the Axial and Sagittal angles on the touchscreen display. The App performs a display function only, it does not derive the angles displayed on perform any calculations on the data received from the RJB device. While using connected devices, users can hold desired angles on screen or offset the angle by using the buttons on screen.

The RJB App also allows for user customization and personalization. The ability to switch from light mode to dark mode will change the theme from light to dark and can be used if lower brightness display is preferred. There are also options to customize the colors of buttons, backgrounds, and display angles depending on user preference.

Once the devices are paired and the platform customized as desired, the user can select the device in use on the touchscreen. When selected, moving the surgical instrument with RJB device attached will change the angle on display. When the user is done using the devices and application, the app can be closed. Closing the app will sever the live connection with the Bluetooth devices but will attempt to re-pair if/when the devices are in range and the application is restarted.

### Firmware

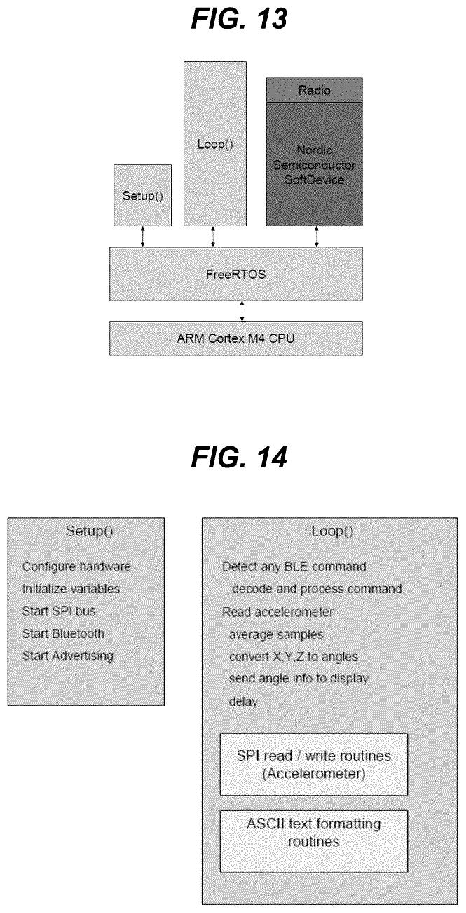

The RJB device utilizes a Bluetooth protocol stack (SoftDevice) provided by the silicon manufacturer, Nordic Semiconductor, and the open-source FreeRTOS real-time operating system (RTOS). The Nordic SoftDevice is provided as a compiled object module library which is linked with the RTOS and RJB-specific software to produce the executable code. In addition, the executable code contains a serial-port-based bootloader, which is a residue of the software development environment and is not used in the field. (See FIG. 13.)

The RTOS “owns” the CPU hardware and provides task scheduling and I/O services to the SoftDevice and RJB software. In a similar manner, the SoftDevice owns the radio hardware and provides Bluetooth services to the RJB software.

The RJB firmware code consists of two major parts: a “setup” routine that is run once at initialization (power-up) and a “loop” that is run continuously after the setup routine completes. (See FIG. 14.)

The RJB setup routine initializes variables and configures the CPU I/O pins and the on-chip SPI interface used to communicate with the accelerometer. The setup routine initializes the Bluetooth channel (device name, power level, etc.) and starts Bluetooth advertising.

The RJB loop routine runs whenever the CPU is not executing RTOS or SoftDevice code. The loop is responsible for reading the accelerometer, computing the angles, and sending this information to the display device. It implements a software delay (RTOS call) to throttle the data gathering process to a rate of two updates per second. Additionally, the loop handles any back channel communication from a paired device. This feature is currently only utilized during manufacturing test and calibration. No back channel commands are currently used in the field with the display device (tablet).

Included in the RJB software are routines to perform SPI read and write operations and ASCII text numeric formatting routines (word-to-BCD, word-to-HEX, etc.).

The RTOS is responsible for setting up the execution environment and providing a small number of low-level services to the RJB-specific code. Among these are pin input/output, time delays, and SPI read and write routines. The correct operation of these is easily determined during software testing.

The Bluetooth SoftDevice is widely used in the industry and is used as-is from Nordic Semiconductor. The RJB uses a small subset of the SoftDevice to provide the UART emulation (read, write) needed to send data to the display device.

### App Software

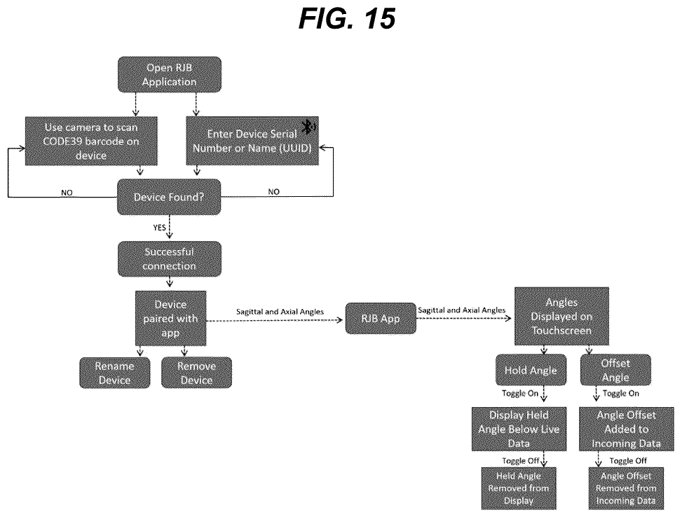

The architecture design chart for the RJB App is depicted in FIG. 15. The App connects to an RJB device via either manual serial number entry or automated barcode scanning. Once paired, the App displays the Axial and Sagittal angles communicated by the RJB onto the tablet screen, with the ability to hold or offset the displayed angle.

### Software Development Environment

The software development environment is a Windows™-based PC running the Arduino integrated development environment (IDE) with the Adafruit Feather nrf52832 as the target execution vehicle. Adafruit provides board support libraries for the Feather, and Nordic Semiconductor provides the Bluetooth protocol stack (SoftDevice) for the nrf52832 as an executable binary. The Arduino IDE automates the compile-link-download process combining the RJB code with the Arduino and Adafruit libraries and the Nordic SoftDevice, and downloading the code using an Adafruit USB boot loader resident on the Feather board.

Source code is written in either “C” or “C++”. The compiler, linker, and related programs are GNU gcc as provided in the Arduino IDE.

Initial software development was done on the Adafruit Feather nrf52832 board. Example programs provided by Adafruit were used as basis of the Bluetooth communication between the RJB and display device. The Adafruit “Bluefruit Connect” smart phone ap was used simulate the tablet-based display device.

Once the Bluetooth code was working, the accelerometer was added via a simple daughter board and code was the rest of the RJB code was written and debugged.

Later in the development cycle, custom boards were built that combined all the chips—microprocessor, accelerometer, and USB port—on one board.

Because the RJB device does not include the USB port used on the development boards, the Segger “JLink” tool is used to program the microprocessor through the SWD (single wire debug) port. The J-Link tool can be accessed via the Arduino IDE or by a Windows command-line or batch-file interface. The SWD hardware interface consists of 10 PCB contacts on the back side of the RJB board. Production programming is done via a fixture the combines the Segger J-Link tool with a custom-designed spring-loaded pin matrix to access the SWD pads on the back of the RJB.

### App Software

The RJB App was written in the Swift coding language, on both iOS and Android platforms.

### Maintenance Procedures and Life Cycle Model

Maintenance may occur on an as needed basis defined by testing for Configuration and Risk management. The app may remain on the distribution platform and updates may be delivered through the respective platforms. Users need not be notified of updates but they may be made available when deployed.

Testing of the app may be performed on a frequent and efficient basis to ensure it is operating properly and as designed:
General Usage—Quarterly—A step by step test of all functions in the app to ensure that they are operating properly and as designed.
Configuration—Quarterly—Internal dive into the coding to ensure there are no newly developed bugs or glitches resulting in issue for the use of the app.
Risk Management—Quarterly—Confirmation and inspection of code to ensure no data will be saved and that usage of the app will not result in increased device risk.

Users may have the ability to download the application from the Apple App Store or Android App Store. The RJB app may remain on the App Store through updates and bug fixes unless otherwise noted.

If there is a time the application is set to be retired, users may or may not be notified. Application will be removed from distribution platforms and any maintenance or updates will cease.

### Configuration Management Plan

Configuration management of RJB may occur on a frequent and efficient basis.

### Test Frequency

Testing may occur on a quarterly basis OR upon receipt of user inquiry or concern. A general overview of the testing process is as follows. Use the entire application as intended, connecting between 1-4 devices and monitoring angles displayed on screen. If configuration of application is incorrect in any way, the test will result in a failure. If the configuration does not affect the performance of the app, modify existing risks, or add new risks, the application will continue to be available for use and update will be sent to distributor platform for the user to download. If the application configuration does affect the app performance or modifies the app risk profile, a risk evaluation may be performed and any configuration changes may be evaluated as necessary before the update is released to users.

### Risk (Hazard) Management Plan

The RJB app may be continuously monitored and tested to confirm the risk of usage remains low per the software hazard analysis. Combining frequent checks and user feedback, the app will consistently be required to pass risk evaluations. Risk evaluations will ensure the user or any subjects/patients will not come to harm through use of the application.

### Cybersecurity

The cybersecurity risks associated with the RJB may be evaluated through the following:
Identification of assets, threats, and vulnerabilities;
Assessment of the impact of threats and vulnerabilities on device functionality and end user/patients;
Assessment of the likelihood of a threat and of a vulnerability being exploited;
Determination of risk levels and suitable mitigation strategies;
Assessment of residual risk and risk acceptance criteria.

The cybersecurity risk analysis performed, the controls identified, the comparison with the National Vulnerability Database, and the labeling information detailed within the Cybersecurity Assessment demonstrate that all risks associated with network capable interfaces of the device have been mitigated to the lowest possible level. The device meets all regulatory requirements pertaining to cybersecurity and wireless communication.

### Angle Measurement Accuracy

In order to verify the system-level angle measurement accuracy of the RJB, the angular outputs of the device were compared with a NIST-traceable calibrated digital protractor mounted at the same axial or sagittal angle in space as the RJB. The RJB was inserted into the instrument handle slot with the instrument held in a vice at various axial and sagittal angles. The instrument used for testing was a gearshift probe with a plastic handle manufactured with a slot to accommodate the RJB. Both the handle material and instrument type are representative of an instrument commonly used in lumbar pedicle screw procedures. The RJB was oriented during testing such that the sagittal angle of the device was zero when axial angles were measured, and vice versa. To perform the test, the instrument was positioned at a given angle with the RJB in either the axial or sagittal orientation and held in place. The angle displayed by the RJB app was recorded, along with the angle displayed by the digital protractor that was aligned with the shaft of the instrument, parallel to the RJB. The instrument angles were chosen at random spanning an entire 0-90° of the RJB's working range.

All axial and sagittal angles measured by the RJB fell within the system-level accuracy requirement of ±1° compared to the digital protractor reference. The system-level accuracy acceptance criteria for the RJB was changed to ±1°. A reference to ±3.5° criteria remains in the test report and protocol as that was the criteria at the time of testing. See Table 3 below for a summary of the test results.

TABLE 3
Angle Measurement Accuracy Testing Results

| | Axial Orientation | Sagittal Orientation |
|---|---|---|
| # RJB Devices Tested | 34 | |
| # RJB Measurements | 102 | 102 |
| Made (total) | | |
| # RJB Measurements | 102 | 102 |
| Within ±3.5° Acceptance | | |
| Criteria | | |
| Average Difference | 0.36 ± 0.32° | 0.74 ± 0.47° |
| Between RJB and | | |
| Protractor | | |
| Measurement ± stdev. | | |

These results demonstrate the safety and effectiveness of the RJB operation in measuring axial and sagittal angles of the device relative to gravity.

### Battery Life Testing

Battery life testing was performed following distribution and accelerated aging validation to confirm that these activities do not adversely affect the RJB's 8-hour usage life. Devices returned from distribution and accelerated aging testing were removed from their sterile packaging unaltered, and turned on by removing the battery tab. The activated devices were then monitored for on/off status until automatically shutting off after 10 hours had elapsed as designed. See Table 4 below for a summary of the test results.

TABLE 4
Battery Life Testing Results

| | Post-Distribution | Post-Accelerated |
|---|---|---|
| | Testing | Aging Testing |
| # RJB Devices Tested* | 25 | 20 |
| # RJB Devices Meeting 8- | 25 | 20 |
| hour Acceptance Criteria | | |

### Performance Testing—Non-Clinical

All devices demonstrated a battery life of 8 hours, meeting the acceptance criterion.

### Usability Testing (Surgeon Evaluation)

Usability testing in the form of a surgeon evaluation of the RJB was performed to validate the design of the RJB device and application. The test was also intended to determine the effectiveness of device labeling in instructing the users on proper device setup and operation. Fifteen surgeons evaluated the device after being trained on the setup and operation of the RJB. Surgeons were also provided a copy of a Surgical Technique document to read. Following training, surgeons were instructed to set up and operate the RJB according to the Surgical Technique and complete the steps outlined in the User Instructions document accompanying the test. Surgeon feedback was captured with a Usability Test Questionnaire following the test.

All users agreed that the RJB device fit snugly into the instrument handle and that the battery pull tab was easy to remove. The majority of users also had no negative feedback on the use of the barcode scanning feature and agreed that it was easy to pair the RJB device and app. All surgeons who were able to test the Hold and Offset features reported that they worked properly and without error and that the device labeling was clear and helpful. All users agreed or strongly agreed that the ±3.5° accuracy is acceptable for clinical use of the RJB.

### Procedural Accuracy Testing

Procedural accuracy testing of the RJB was performed in order to validate the accuracy of the device under simulated use conditions.

The procedural accuracy testing was performed on a cadaveric torso that fully contained the lumbar anatomy. The cadaver was placed on a table and draped to represent the OR environment. Additionally, imaging equipment including fluoroscopy was included as is typical for pedicle screw procedures.

Sixteen users took part in the test, all surgeons specializing in orthopedic or neurological spine surgery spanning an experience level of 3 to 36 years in practice (mean 15.9 years±stdev 11.5 years). The surgeons were provided with the complete RJB labeling (IFU and Surgical Technique) to read prior to the test, as well as received training by Ruthless personnel on device operation.

As part of the test, MRI images were taken of the cadaver prior to the test in order to determine clinically relevant axial target angles to be used during testing. A frozen cadaver torso (containing the entire torso from the base of the neck to the upper thigh/femur levels) was placed into a 1.5 T closed MRI scanner (GE Signa EXCITE 1.5T MRI Scanner upgraded with 1.5T SIGNA™ HDxt SIGNA™Works Edition software) in a supine position as per standard protocol with a live patient. The standard protocol used by the MRI scanner for lumbar spine as per the software upgrade was used for the lumbar spine of the cadaver, which included axial and sagittal images that included the entire lumbar spine as well as the T12 and sacral area. Axial T2 weighted images were used for determining the clinically-appropriate axial angles for the test.

Briefly, the test method involved the user positioning the RJB instrument against a pedicle to simulate pedicle screw placement. The instrument was positioned in a composite angle using the pre-planned axial angle from MRI imaging and sagittal angle from fluoroscopy imaging. When the instrument was positioned at the desired composite angle, both a fluoroscopic and a pair of photographic images were taken of the instrument to allow for angle calculation following the test. The surgeon then places the instrument down, picks it up again and repeats this process two more times at the same pedicle. This entire process is repeated for each of the 5 levels of the lumbar spine.

Following the test, the images taken were used to calculate composite angle error as compared to the ground-truth measurement (gravity, through the use of a plumb line). See subsection below for additional detail of how composite angles were calculated.

### Composite Angle Calculation

The composite angle, shown in FIG. 16 as C, produces an axial projection A and the sagittal projection B.

The axial angle A and sagittal angle β are deviation from vertical. The composite vector deviates from vertical by angle C. The relationship between the angles is:

tan 2(C)=tan 2(A)+tan 2(B)

For the purposes of this test, there are two composite vectors-one determined by the axial and sagittal angles displayed by the RJB, the other is computed from the axial and sagittal angles measured in the camera images. The task is to measure the difference between these two composite vectors.

The vertical axis of the coordinate system shown in FIG. 16 may be aligned to the composite vector C computed from the camera images and the angles A and B represent the angular error between the RJB display values and the camera image value. The vector C will now represent the composite error between the display value and the camera value. The equation shown above can be applied to compute the composite angular error.

From the images taken during the test, the actual axial and sagittal angles were measured. These were compared to the RJB display angles and the angular error in each axis was computed. The composite error was computed using the formula:

Ec=tan−1(SQRT(tan(Ea)2+tan(Es)2)

Where Ec is the composite error, and Ea and Es are the axial and sagittal errors, respectively.

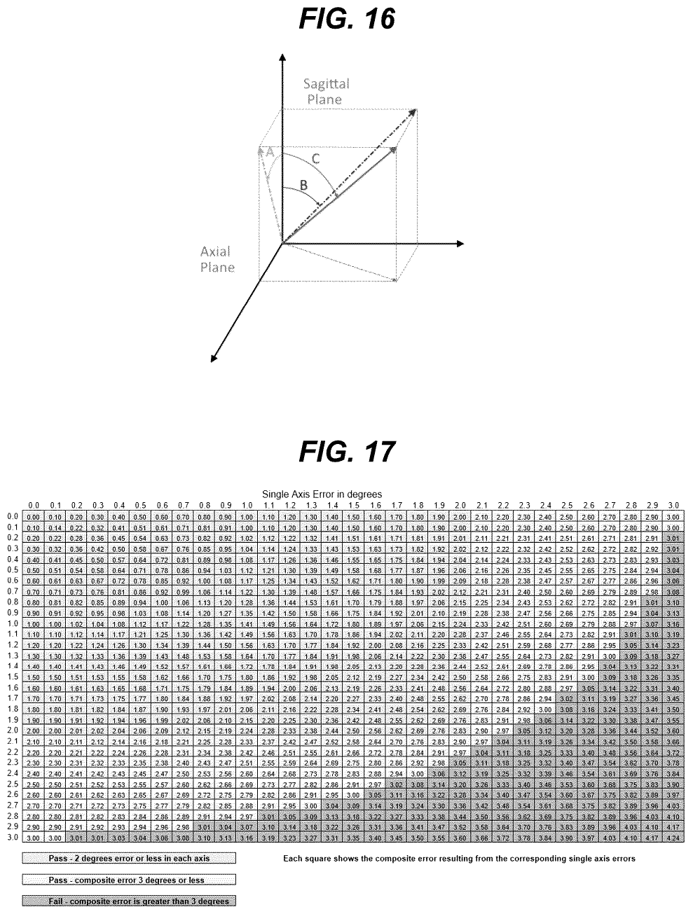

Shown in FIG. 17 is a table showing all possible axial and sagittal errors in the range of 0 to 3 degrees and the corresponding composite errors that result. The 3-degree composite error limit is shown as middle region of FIG. 17. This was used for the pass/fail determination. The rectangular region in the upper left represents the alternative pass/fail criterion of 2 degrees of error in either axis. The region in the lower right of FIG. 17 represents a “fail” where the composite error is greater than 3 degrees.

The acceptance criteria of the test included both the composite angle error of the photographically-measured angles as well as the error between fluoroscopically-measured angles:

Axial angles displayed on the RJB app may be within ±3° (absolute error of the composite angle) OR ±2° (absolute error of the individual component) of the measured angles from the pictures.

Sagittal angles displayed on the RJB app may be within ±3° (absolute error of the composite angle) OR ±2° (absolute error of the individual component) of the measured angles from the pictures.

Sagittal angles from rounds 1, 2, and 3 measured on the fluoroscopic images shall be within ±3° from each other for each compound angle.

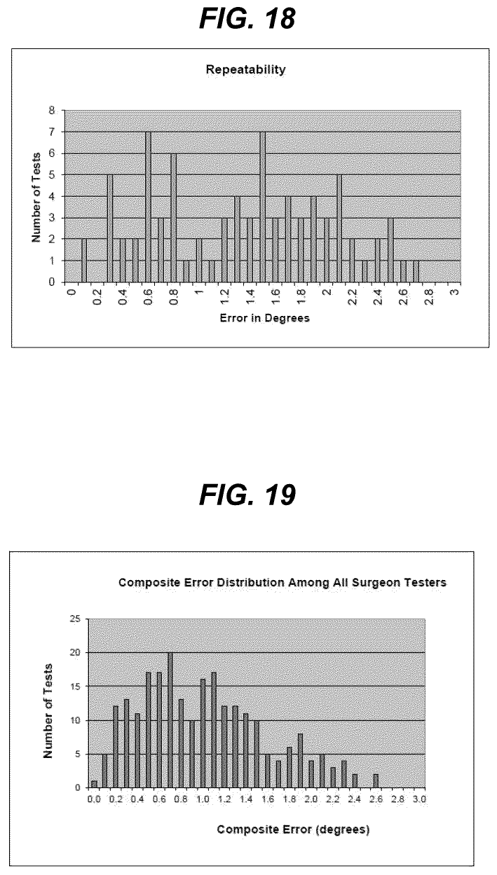

Results for angular composite error, computed from the square root of the sum of the squares of the axial and sagittal angles, is presented in FIG. 18. The distribution of all 240 trials (3 measurements at each of 5 levels for 16 surgeons) is shown. The majority of composite errors are in the 0 to 1.5 degree range, with larger errors trailing off rapidly.

Repeatability of the fluoroscopic angles was computed from the difference between the largest and smallest fluoroscopic sagittal angle at each level. The results are summarized in FIG. 19 showing all 80 tests (16 surgeons and five levels each).

All sixteen surgeons participating in the tests met the acceptance criteria without exception. None of the composite errors exceeded 2.6 degrees (±3° criteria) and the largest fluoroscopic image repeatability deviation was 2.7 degrees (±3° criteria). The results of this testing validate the procedural accuracy of the RJB device through simulated clinical use.

### Surgical Technique and Instructions for Use

### Device Description

The Ruthless Spine RJB device is an intraoperative surgical angle measurement guide that attaches to surgical instruments to measure the angle of the instrument relative to a vertical plumb line in line with gravity. The device can measure the axial and sagittal angles relative to gravity. The RJB system only provides measurements for angles in two planes relative to the vertical gravitational plumb line. As such, the RJB device does not provide surgical assistance, guidance, or navigation against patient anatomy. The RJB device is not intended to replace a surgeon's clinical judgement and has not undergone clinical evaluation. No clinical benefit has been demonstrated or is claimed.

The RJB device is provided sterile for single use and utilizes Bluetooth Low Energy (BLE) to connect to a tablet computer and display the angle measurements via the RJB Application (App). A set of handles and instruments compatible with the RJB are provided with the device for use in lumbosacral pedicle screw placement. The following components may form parts of the RJB system:
RJB Device
Quick Connect Axial Ratcheting Handle
Straight Probe
Duckbill Probe
RJB Application

A Tablet Computer or other display used to operate the device.

The Ruthless Spine RJB device is intended to measure the angle of surgical instruments in two planes relative to a vertical plumb line in line with gravity. It is indicated for use during lumbosacral pedicle screw implantation in conjunction with applicable spinal instrumentation and as an adjunct to fluoroscopy or intraoperative x-ray. The RJB device is not intended to replace a surgeon's judgment and has not undergone clinical evaluation. No clinical benefit has been demonstrated or is claimed.

### RJB Operating Instructions

1. Insert the RJB device 14 into the handle slot. Follow the markings for device orientation. The slot should only allow the device to be inserted in one way.
2. Turn on the RJB device by removing and discarding the battery pull tab.
3. Deploy the Ruthless RJB app on a tablet.
4. When the app is opened, a tablet screen prompts to pair the RJB device. The RJB serial number can be scanned from the label or manually inputted.
5. After pairing the device, a prompt will appear to select the instrument type.
6. Once the instrument type is selected, the starting screen will appear. From here, the user can see the angle of the device, hold the angle, offset the angle, edit the instrument name, choose display precision, and watch tutorial videos. a. Hold and offset features are controlled via touchscreen buttons in the tablet app.
7. For use, the instrument should be oriented such that the face of the RJB (marked with ‘UP’ and ‘DOWN’ and visible through the front of the handle slot) is in a plane parallel with the plane of the operating table, without introducing rotation about the instrument shaft. During use, the instrument may be tilted in the axial and sagittal planes simultaneously but should not be rotated about its own shaft. Use the marked face of the RJB to reference the position of the instrument throughout the procedure.
8. When using the RJB, it is recommended that the surgeon follow standard fluoroscopy-guided technique, utilizing fluoroscopy as necessary to confirm pedicle screw trajectory intraoperatively. The RJB is a spatial tool and not a navigation system providing trajectory guidance.
9. If Bluetooth connection is lost (displayed angles disappear or freeze), restart app.
a. If RJB device does not pair, turn on, or app does not restart, discard the device by disposing in accordance with facility protocol. Revert to standard fluoroscopy-guided technique to complete the procedure.
10. To terminate operation of the device, close the RJB app. Remove RJB from the instrument handle and dispose in accordance with facility protocol.

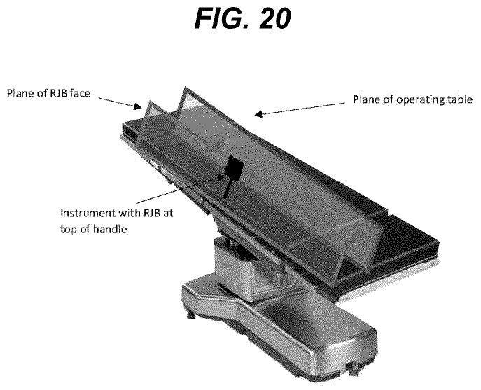

Following insertion of the RJB into a handle, activation, and pairing, orient the RJB instrument in space for proper use. The instrument 12 should be oriented such that the face of the RJB (marked with ‘UP’ and ‘DOWN’ and visible through the front of the handle slot) is in a plane parallel with the plane of the operating table, without introducing rotation about the instrument shaft as shown in FIG. 20. During use, the instrument may be tilted in the axial and sagittal planes simultaneously but should not be rotated about its own shaft. Use the marked face of the RJB to reference the position of the instrument throughout the procedure. See sections below for further detail regarding instrument angles.

The following definitions of the axial and sagittal angles measured by the RJB are presented individually to aid in user understanding. While the axial and sagittal angles are displayed to the user individually, the RJB may be used in positions that contain both axial and sagittal angle components simultaneously, comprising a composite angle.

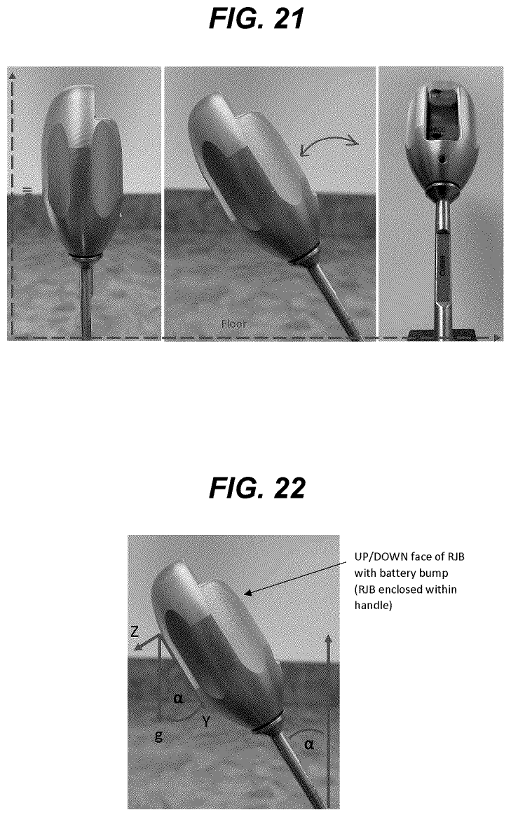

Axial Angle Definition: The axial angle displayed by the app represents the angle of the RJB (and instrument) in space created when the RJB is tilted from vertical such that the face marked with ‘UP’ and ‘DOWN’ points towards the ceiling or floor as shown in FIG. 21. The term ‘axial’ preferably defines the angle of the RJB in space relative to gravity, not relative to the patient or spine.

In FIG. 22, the example axial angle (a) shown is the angle between gravity (g) and the instrument shaft. One direction of the angle will be positive. The opposite direction will negative, if the instrument is tilted axially in the opposite direction while maintaining the same orientation of the RJB face. (Z and Y are internal axis to the device. These are irrelevant to the user.)

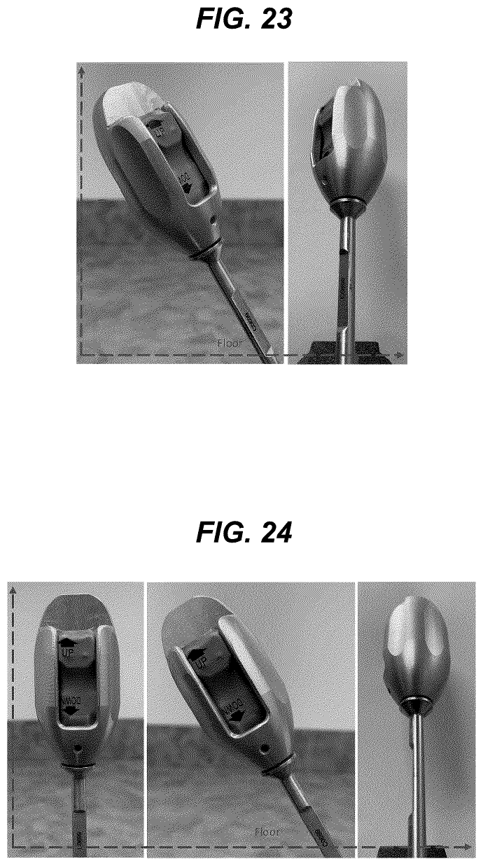

The RJB displays accurate axial and sagittal angle measurements whether the instrument shaft is held in a pure axial orientation (zero sagittal angle component), a pure sagittal orientation (zero axial angle component), or a compound angle (containing both axial and sagittal components simultaneously). In order to ensure accurate measurements, the instrument should be tilted in the axial and/or sagittal planes such that the face of the RJB remains parallel with the plane of the operating table, without introducing rotation about the instrument shaft. See FIG. 23 for an example of incorrect use, orienting the RJB in space while incorporating rotation.

Sagittal Angle Definition: The sagittal angle displayed by the app represents the angle of the RJB (and instrument) in space created when the RJB is tilted from vertical such that the face marked with ‘UP’ and ‘DOWN’ is tilted left or right when facing the user as shown in FIG. 24. The term ‘sagittal’ defines the angle of the RJB in space relative to gravity, not relative to the patient or spine.

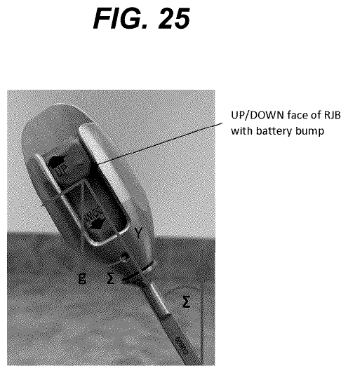

In FIG. 25, the example sagittal (E) shown is the angle between gravity (g) and the instrument shaft. One direction of the angle will be positive. The opposite direction will be negative. (Y is an internal axis to the device. It is irrelevant to the user.)

### Device Function

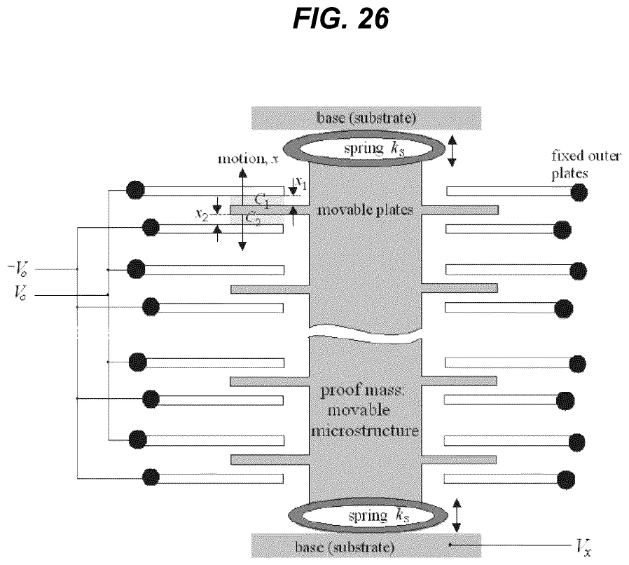

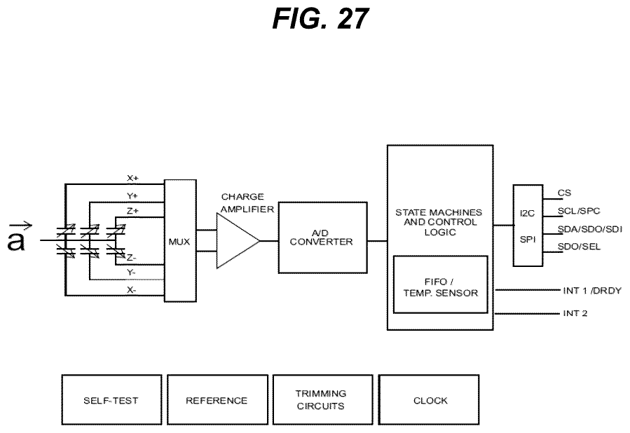

The RJB device measures angular displacement from vertical by using a Micro Electro Mechanical System (MEMS) accelerometer, such as shown in FIG. 26, to measure the gravitational field in all three axis. The three gravitational measurements are converted into angles by basic trigonometry. The displacement angles are communicated to a display device by a Bluetooth Low Energy (BLE) wireless link. The RJB is powered by a 1.5 Volt coin cell.

The MEMS accelerometer uses a spring-supported mass which deflects due to gravity. The displacement is measured through the change in capacitance between plates attached to the mass and stationary plates attached to the substrate. The change in capacitance is converted into units of micro-G's by circuitry on-chip. By using three separate structures, orientated orthogonally, the gravitational field can be measured in all three axis, x, y and z.

In an embodiment, such as shown in FIG. 27, the accelerometer used in RJB is manufactured by ST Electronics, part number LIS3DSH. This is a 3-axis device with 14-bit resolution over a +/−2 g range.

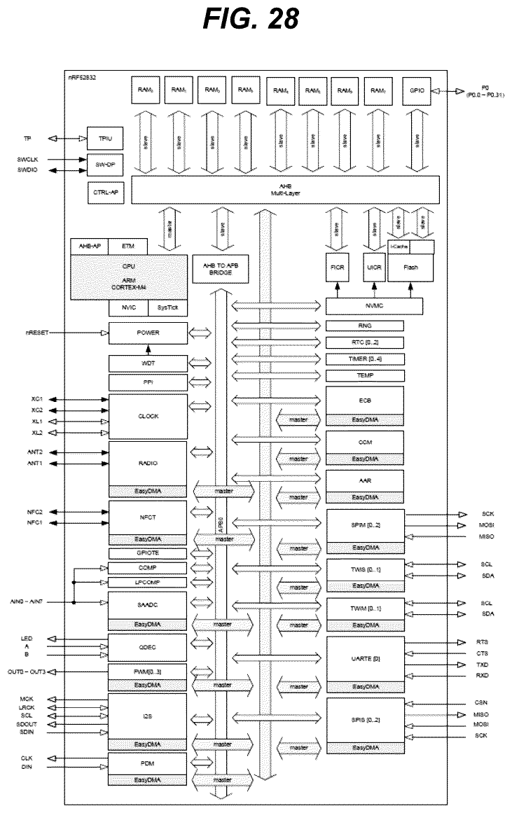

In an embodiment, such as shown in FIG. 28, an integrated microprocessor and RF radio module, Raytac Co. MDBT42Q, is used to control the accelerometer, compute the angles and send the results to the display device. The MDBT42Q module contains a Nordic Semiconductor nRF52832 integrated circuit with an ARM Cortex-M4 32-bit processor, integrated memory, peripherals, and 2.4 GHz transceiver. Managing the accelerometer requires few of the resources of the microprocessor and many of the peripherals shown in the block diagram need not be used.

In addition to the nRF52832 chip, the MDBT42Q module also includes a 32 MHz crystal, a DC/DC converter, and an antenna. Additional components (external to the MDBT42Q module) on the RJB device are a coin cell, a DC/DC up converter, and two status indicator LEDs.

### Coordinate System

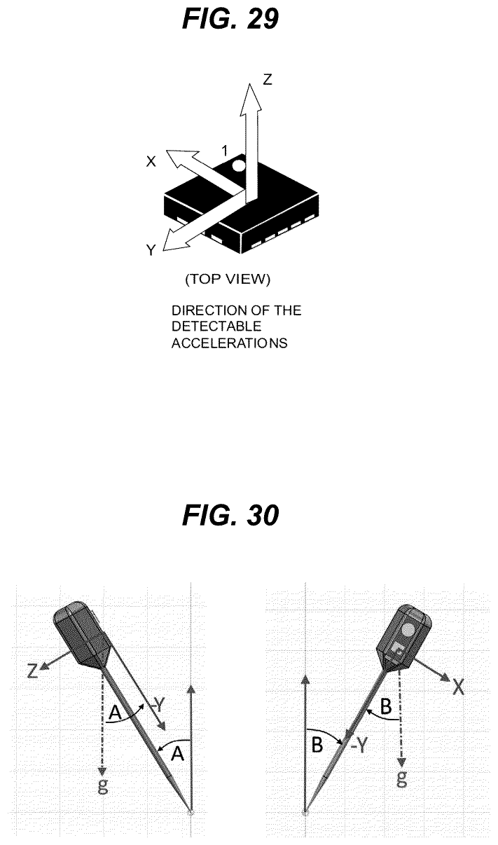

The accelerometer manufacturer may define the X, Y, and Z axis as shown in the diagram, relative to pin 1 of the device—shown as the white dot in FIG. 29.

The accelerometer is mounted on the RJB circuit board as shown. The RJB device connects to the surgical tool handle at the 12 o'clock orientation with the battery facing to the rear as shown in FIG. 8.

In an embodiment, when mounted to an instrument, the coordinates map may be as follows as shown in FIG. 30.

The axial angle (A) is computed by the arcsin of Z divided by g (equal to the maximum value of Z); the transverse angle (B) is the arctangent of (X/Y), which in the example shown above is negative. Axial angles range from 0 to 60 degrees and transverse angles range from −60 to ±60 degrees from vertical. Note that the transverse angle is the deviation from vertical as measured in the plane of the RJB. When the axial angle is non-zero, the transverse angle becomes compound. Under this condition, an observer looking horizontally will see a transverse angle foreshortened by the axial deviation from vertical. To accommodate clinical situations that measure transverse angles from a horizontal perspective, the RJB performs the necessary conversion to display the transverse angle in this form (the sagittal angle displayed on the RJB app).

### Software Implementation Details

1. Because the angle calculation relies on only the ratio of the axis values, the units are Insignificant and the raw binary values from the accelerometer are used in the calculations.
2. Since the RJB is measuring only the gravitational field of the earth (nominally 1 “G”), only half of the accelerometer's dynamic range (in its most sensitive setting, +/−2G) is used and successive readings show variations due to noise. To filter the results multiple accelerometer readings are summed together effectively computing the average value of each axis.
3. The three axes of the accelerometer may not have identical span (max vs. min) and the zero-g values may not be zero. Accordingly the software normalizes the three channels in span and offset prior to doing the trigonometric calculations. This process relies on calibration constants determined during manufacturing test.
4. The angles computed from the accelerometer data are normalized using gain and offset constants determined during calibration.
5. The transverse angle values are foreshortened through a trigonometric relationship with the axial angle to convert the values into the equivalent angle measured from the horizontal line of sight (sagittal angle).
6. To minimize the power consumption, the trig functions were implemented through sine, cosine, and tangent look-up tables. The arctangent and arcsine are found by searching the tangent and sine tables for the value. Once the closest entry is located, address of that location in the table is the angle. The tables are indexed in 0.1 degree increments. To minimize the number of table accesses to locate, supplementary 10 degree “coarse” resolution and 1 degree resolution “medium” tables are implemented for arcsine and arctangent searching. The coarse table provides the 10 degree starting point in the medium table, which in turn provides a 1 degree starting point for the search in the actual table. The average search is accomplished in 15 accesses (5 in each table).
7. The Bluetooth Low Energy (BLE) capability was obtained from an object-code library (“soft device”) supplied by the chip manufacturer, Nordic Semiconductor. No modifications were done to this library. The RJB device Implements the BLE UART function to transmit short ASCII text strings with the Alpha and Beta angles. Current software issues such a text string twice a second.

### Calibration

After the RJB device is assembled, the accelerometer is calibrated to compensate for any errors induced during assembly. Such errors can be caused by the mechanical stress of being soldered or from board-to-board variations in the physical placement of the accelerometer on the board.

The raw output of each channel of the accelerometer is normalized to compensate for gain and offset errors in each channel as well as channel cross-talk (variations in one channel adversely affecting the value of another). This is accomplished by placing the assembled RJB on a gimbaled calibration jig and precisely rotating the RJB orthogonally through the angle extremes as shown in FIG. 8.

Each of these orientations maximizes one axis (with either positive of negative polarity) while minimizing the other two. From these measurements the dynamic range, or span, and the zero-g offset of each channel can be computed.

The spans of each channel are typically unequal and not symmetric. Since the trigonometric relationships rely on ratios between the axes, the spans must be normalized. The Y axis is chosen as the reference and X and Z are scaled to match.

The accelerometer also demonstrates different zero-g offsets among the channels. FIG. 9 shows the effect rotating a typical accelerometer in one axis has on the other two—in this case from position “0” to position “1” of FIG. 8.

In both position “P0” and position “P1” the accelerometer's X-axis and Z-axis are horizontal (perpendicular to gravity) and should indicate zero. The X's and Z's are the actual values and represent an offset error.

Position “2” and “3” produce another such graph, plotting Y and Z against the horizontal axis X. This will produce two more offset error values (for Y and Z). The last two positions, “4” and “5”, will plot X and Y against Z and provide two additional offset values (for X and Y) for a total of twelve offset values—four for each axis. Since none of these are more significant than the others, a simple averaging of the four offsets for each axis provides a single value. This is called the static offset for that channel.

Since the slopes of the lines in FIG. 9 are non-zero, there is also a dynamic offset which is a function of the perpendicular axes values. This is essentially “cross talk” between one channel and the other two and can be expresses as a gain. In FIG. 8 it is shown as Xgain_y (the offset in X due to the Y-axis value) and Zgain_y (the offset in Z caused by Y). These gains are applied to the static offset values of the perpendicular channels to adjust the static offset previously described. As a result, the offset for the X-axis, for example, is X_offset=Xstatic_offset+(Ystatic_offset*Xgain_y)+(Zstatic_offset*Xgain_z). The offset is subtracted from the raw accelerometer value for each axis. The resulting values for X and Z are then normalized for the span of Y at which point they are used to compute the angles.

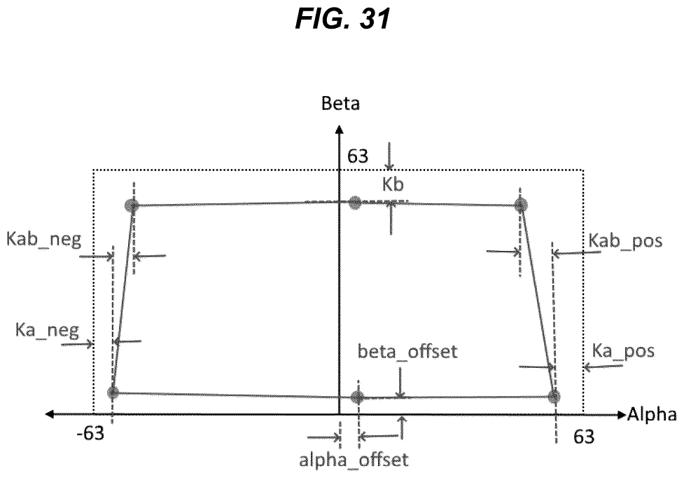

The accelerometer normalization process may not remove all the error, and the angles computed may be adjusted through a second normalization process to improve accuracy. To do this, the RJB is precisely positioned at the extreme corners of its calibration range, that being 0 to 63 degrees in beta and ±63 to −63 degrees in alpha-represented by the dotted line rectangle FIG. 31. The 63 degree calibration range was chosen to provide some guard band with the 60 degree operating range specification and because of the 200 step/revolution drive motors in the gimbal mechanism used to manipulate the RJB during calibration favor angles divisible by 9 or even fractions thereof.

The angles measured at each of these positions, represented by the green dots, are compared to the gimbal angles and the angle offset and gain adjustments are computed to essentially “stretch” the red polygon shown above to match the dotted rectangle. For the alpha axis, the gain adjustment are in both as a function of alpha (Ka, above) and as a function of the product of alpha and beta (Kab, above). For the alpha axis, separate Ka and Kab gain adjustment values are computed for positive and negative alpha angles to accommodate any asymmetry that might be present.

As an additional calibration step, the gimbal is used to sequentially position the RJB through a fine grid of angles (currently 4.5 degrees per step) on the over the entire full range of both axes (0 to 63 degrees in beta and −63 to 63 degrees in alpha). The computed values are compared with the gimbal angles and an error value is calculated for each point. The error value at each point is stored in a two-dimensional array and used as a correction factor for subsequent calculations.

As a final step, a “verify” cycle is performed. The RJB is sequenced through the same range of angles and the error between the corrected angle and gimbal angles are recorded. The RJB is determined to “pass” if none of the errors exceed an upper limit (currently 0.3 degrees).

### Cybersecurity and Controls

### 1. Access Control

a. Control: Device is labeled for prescription use only. RJB app is unusable without a paired RJB device, limiting use to physicians in possession of a prescription RJB.
b. Control: RJB/app pairing requires entry of the RJB serial number (available only on device packaging/labeling) into the app.
c. Control: The RJB is only accessible via the Bluetooth connection, there are no wired interfaces. Access is physically limited by the Bluetooth range of RJB.

### 2. Encryption

a. Control: None, no patient-identifiable or personal health data is utilized or transmitted by the RJB.

### 3. Intrusion Detection/Prevention

a. Control: RJB firmware and app software are not connected to any network.
b. Control: RJB and app access is limited by pairing functionality. Once an RJB is paired with the RJB, the app cannot pair. In this scenario, the app would not display any angular values and the RJB would be determined to be nonfunctional and discarded/not used.
   4. Lack of Data Security, Especially Susceptibility for Falsification, Unwanted Interaction with Other Programs, or Viruses
a. Control: Communication with RJB firmware is limited to backchannel commands for either 1) turning off update messages or 2) data rounding. Turning off update messages would be interpreted by the app as an unusable/defective device. Turning off rounding would not affect data accuracy and therefore not affect user or patient safety.
b. Control: The RJB cannot be recalibrated via backchannel commands.
c. Control: Undetected manipulation of angle values in the RJB app would be physically limited by requiring direct visual access to the surgical procedure.
d. Control: The RJB's labeling instructs the user to not download other apps for use on the tablet nor connect the tablet to any network during RJB app use so that no other insecure code could compromise the tablet.
e. Control: The RJB app was written using proper coding standards, allowing it to be sandboxed once installed on the tablet. Sandboxed means that reasonable measures have been taken by the tablet manufacturer to isolate apps so that they can access only certain resources, programs, and files within the tablet computer system (iOS & Android). No extra applications may directly interfere with RJB app, ensuring that all app data and remote access is protected.
f. Control: The RJB's labeling instructs the user to turn on Do Not Disturb Mode on the tablet to mitigate indirect interference by other applications (displaying popup notifications) during use.
g. Control: To mitigate Wi-Fi vulnerability, the RJB's labeling instructs the user to always have Wi-Fi turned off while using the app.
h. Control: To mitigate Bluetooth and Wi-Fi vulnerability, the RJB's labeling instructs the user to ensure all OS security updates are installed on the tablet prior to app use. The labeling also instructs the user to turn off all other Bluetooth devices in the operating room (use environment) during RJB pairing with the tablet.
i. Control: To mitigate USB vulnerability, the RJB's labeling instructs the user to not allow the tablet to be plugged into any external USB computers, networks, or other peripherals during use. Only factory delivered charging cables and adapters should be used.
   5. Software Integrity from Point of Origin to Distribution from Manufacturer
a. Control: RJB firmware is programmed onto the RJB device at the time of manufacturing and is not accessible or alterable by the user following initial programming.
b. Control: RJB app software is stored at the point of origin within a two-step verified private password protected code repository with a limited number of developers having access. All code is reviewed internally at the point of origin before compilation for any erroneous code or security issues. The code was minimized to make code review efficient when checking for any added malicious code.
c. Control: Once compiled, the iOS version of the application is uploaded to the iOS app store for iOS distribution. The iOS version is maintained on an official Apple Ruthless Spine developer account and is reviewed by Apple for any apparent malicious code or coding guideline infractions before being posted to the store for download by RJB reps or the healthcare provider. The Android version of the app bypasses the Google Play store and is provided directly to the doctor or representative by a time limited one-way download link that is unique to the specific user. iOS and Android also sandboxed the RJB app from the OS and other apps, and the RJB app does not require internet access for use.
d. Control: To ensure validated updates and patches for the app, all developer code will be thoroughly reviewed and tested using all defined V&V tests when any code is added or changed. Tablet operating system (OS) security updates will come directly from the OS developers and will address any new security threats to Wi-Fi & Bluetooth.

The current invention provides significant benefits. For example, the system 10 and its associated methods help the formation of pilot holes and the implantation of pedicle screws at the desired trajectory. This leads to more successful spinal surgeries. The current invention also provides a factor of repeatability, which is important because multiple pedicle screws are generally implanted in spinal surgery. The current invention also help avoid the breaching and/or perforation of the lateral or medial cortical walls, which could result in significant spinal cord injury or other complications.

As noted above, the current invention is not limited to spinal surgery. Indeed, the current invention may be used in other medical procedures where the trajectory of surgical instruments or tools and/or other types of implants is desired.

It is understood that any aspect and/or element of any embodiment of the system 10 described herein or otherwise may be combined with any other aspect and/or element of any other embodiment described herein or otherwise in any way to form additional embodiments of the system 10 all of which are within the scope of the system 10.

Where a process is described herein, those of ordinary skill in the art will appreciate that the process may operate without any user intervention. In another embodiment, the process includes some human intervention (e.g., a step is performed by or with the assistance of a human).

As used herein, including in the claims, the phrase “at least some” means “one or more,” and includes the case of only one. Thus, e.g., the phrase “at least some ABCs” means “one or more ABCs”, and includes the case of only one ABC.

As used herein, including in the claims, term “at least one” should be understood as meaning “one or more”, and therefore includes both embodiments that include one or multiple components. Furthermore, dependent claims that refer to independent claims that describe features with “at least one” have the same meaning, both when the feature is referred to as “the” and “the at least one”.

As used in this description, the term “portion” means some or all. So, for example, “A portion of X” may include some of “X” or all of “X”. In the context of a conversation, the term “portion” means some or all of the conversation.

As used herein, including in the claims, the phrase “using” means “using at least,” and is not exclusive. Thus, e.g., the phrase “using X” means “using at least X.” Unless specifically stated by use of the word “only”, the phrase “using X” does not mean “using only X.”

As used herein, including in the claims, the phrase “based on” means “based in part on” or “based, at least in part, on,” and is not exclusive. Thus, e.g., the phrase “based on factor X” means “based in part on factor X” or “based, at least in part, on factor X.” Unless specifically stated by use of the word “only”, the phrase “based on X” does not mean “based only on X.”

In general, as used herein, including in the claims, unless the word “only” is specifically used in a phrase, it should not be read into that phrase.

As used herein, including in the claims, the phrase “distinct” means “at least partially distinct.” Unless specifically stated, distinct does not mean fully distinct. Thus, e.g., the phrase, “X is distinct from Y” means that “X is at least partially distinct from Y,” and does not mean that “X is fully distinct from Y.” Thus, as used herein, including in the claims, the phrase “X is distinct from Y” means that X differs from Y in at least some way.

It should be appreciated that the words “first,” “second,” and so on, in the description and claims, are used to distinguish or identify, and not to show a serial or numerical limitation. Similarly, letter labels (e.g., “(A)”, “(B)”, “(C)”, and so on, or “(a)”, “(b)”, and so on) and/or numbers (e.g., “(i)”, “(ii)”, and so on) are used to assist in readability and to help distinguish and/or identify, and are not intended to be otherwise limiting or to impose or imply any serial or numerical limitations or orderings. Similarly, words such as “particular,” “specific,” “certain,” and “given,” in the description and claims, if used, are to distinguish or identify, and are not intended to be otherwise limiting.

As used herein, including in the claims, the terms “multiple” and “plurality” mean “two or more,” and include the case of “two.” Thus, e.g., the phrase “multiple ABCs,” means “two or more ABCs,” and includes “two ABCs.” Similarly, e.g., the phrase “multiple PQRs,” means “two or more PQRs,” and includes “two PQRs.”

The present invention also covers the exact terms, features, values and ranges, etc. in case these terms, features, values and ranges etc. are used in conjunction with terms such as about, around, generally, substantially, essentially, at least etc. (i.e., “about 3” or “approximately 3” shall also cover exactly 3 or “substantially constant” shall also cover exactly constant).

As used herein, including in the claims, singular forms of terms are to be construed as also including the plural form and vice versa, unless the context indicates otherwise. Thus, it should be noted that as used herein, the singular forms “a,” “an,” and “the” include plural references unless the context clearly dictates otherwise.

Throughout the description and claims, the terms “comprise”, “including”, “having”, and “contain” and their variations should be understood as meaning “including but not limited to”, and are not intended to exclude other components unless specifically so stated.

It will be appreciated that variations to the embodiments of the invention can be made while still falling within the scope of the invention. Alternative features serving the same, equivalent or similar purpose can replace features disclosed in the specification, unless stated otherwise. Thus, unless stated otherwise, each feature disclosed represents one example of a generic series of equivalent or similar features.

The present invention also covers the exact terms, features, values and ranges, etc. in case these terms, features, values and ranges etc. are used in conjunction with terms such as about, around, generally, substantially, essentially, at least etc. (i.e., “about 3” shall also cover exactly 3 or “substantially constant” shall also cover exactly constant).

Use of exemplary language, such as “for instance”, “such as”, “for example” (“e.g.,”) and the like, is merely intended to better illustrate the invention and does not indicate a limitation on the scope of the invention unless specifically so claimed.

While the invention has been described in connection with what is presently considered to be the most practical and preferred embodiments, it is to be understood that the invention is not to be limited to the disclosed embodiment, but on the contrary, is intended to cover various modifications and equivalent arrangements included within the spirit and scope of the appended claims.

What is claimed is:

1. A method for using a handheld surgical instrument, comprising a measurement sensor assembly that is attached to the handheld surgical instrument, that includes an accelerometer which is referenced to an X-, Y-, and Z-axis, which measures displacements of the handheld surgical instrument along the X-, Y-, and Z-axis in relation to gravity, and which provides angular orientation information based on the displacement measurement, the method comprising:
aligning the Y-axis of the measurement sensor assembly with the patient's anteroposterior axis;
aligning the X-axis of the measurement sensor assembly with the patient's craniocaudal axis;
tilting the handheld surgical instrument to a first axial angle without rotating the measurement sensor assembly about its Y-axis;
tilting the handheld surgical instrument to a first sagittal angle without rotating the measurement sensor assembly about its Y-axis;
using the measurement sensor assembly to measure a first X displacement value, a first Y displacement value, and a first Z displacement value;
calculating a first axial angle value using only the first Z displacement value and gravity; and
calculating a first sagittal angle value using the first X displacement value and the first Y displacement value.

2. The method of claim 1 further comprising:
using an imaging system to measure a second sagittal angle with respect to the patient's anteroposterior axis;
foreshortening the first sagittal angle value by the first axial angle value to determine a foreshortened sagittal angle value; and
correlating the second sagittal angle with the foreshortened sagittal angle to determine if the second sagittal angle substantially matches the foreshortened sagittal angle.

3. The method of claim 2 further comprising:
in response to a determination that the second sagittal angle substantially matches the foreshortened sagittal angle, then enabling further use of the measurement sensor assembly via an application; and
in response to a determination that the second sagittal angle does not substantially match the foreshortened sagittal angle, then disabling further use of the measurement sensor assembly via the application.

4. The method of claim 2 wherein the imaging system includes a fluoroscope system.

5. The method of claim 1 further comprising:
aligning the anteroposterior axis of the patient with gravity.

6. The method of claim 1 wherein the accelerometer includes a tri-axial accelerometer.

7. A method comprising:
providing a sensor adapted to take angular displacement measurements relative to a three-dimensional X, Y, and Z coordinate system;
aligning a Y-axis of the sensor with gravity;
aligning an X-axis of the sensor with a craniocaudal axis of a patient;
tilting the sensor to a first sagittal angle and to a first axial angle without rotating the sensor about the Y-axis;
using the sensor to measure a first sagittal angle and a first axial angle of the patient, each with respect to gravity;
using an imaging system to measure a second sagittal angle of the patient with respect to an anteroposterior axis of the patient;
foreshortening the first sagittal angle by the first axial angle to determine a foreshortened sagittal angle; and
correlating the second sagittal angle with the foreshortened sagittal angle to determine if the second sagittal angle substantially matches the foreshortened sagittal angle.

8. The method of claim 7 further comprising:
in response to a determination that the second sagittal angle substantially matches the foreshortened sagittal angle, then enabling further use of the sensor via an application; and
in response to a determination that the second sagittal angle does not substantially match the foreshortened sagittal angle, then disabling further use of the sensor via the application.

9. A method of calibrating a measurement sensor assembly that includes an accelerometer which takes X axis measurement data, Y axis measurement data and Z axis measurement data, the method comprising:
loading the measurement sensor assembly into a calibration device that positions the accelerometer at different angular orientations;
using the calibration device to position the measurement sensor assembly at first angular orientations, to cause a full-scale reading on the X axis while causing a near-zero reading on the Y axis and the Z axis, to cause a full-scale reading on the Y axis while causing a near-zero reading on the X axis and the Z axis, and to cause a full-scale reading on the Z-axis while causing a near-zero reading on the X axis and the Y axis;
taking first raw measurement data for each of the X axis, Y axis and Z axis at each of the first angular orientations;
comparing the first raw measurement data for each of the X axis, Y axis and Z axis at each of the first angular orientations to theoretical ideal values to determine error correction factors for each of the X axis, Y axis and Z axis;
using the error correction factors for each of the X axis, Y axis and Z axis to correct the first raw measurement data for each of the X axis, Y axis and Z axis resulting in first corrected measurement data for each of the X axis, Y axis and Z axis;
using the calibration device to position the measurement sensor assembly at second angular orientations that represent one or more operational range extremes of the measurement sensor;
taking second raw measurement data for each of the X axis, Y axis and Z axis at each of the second angular orientations and using the error correction factors for each of the X axis, Y axis and Z axis to correct the second raw measurement data for each of the X axis, Y axis and Z axis resulting in second corrected measurement data for each of the X axis, Y axis and Z axis;
determining initial sagittal angle α data and initial axial angle β data using trigonometric relationships applied to the second corrected measurement data for each of the X axis, Y axis and Z axis;
comparing the initial angle sagittal angle α data and the initial axial angle β data to theoretical ideal values to determine error correction factors for the initial sagittal angle α data and for the initial axial angle β data; and
storing the error correction factors in memory.
